(19) United States
(12) Patent Application Publication
(10) Pub. No.: US 2009/0306149 A1
(43) Pub. Date: Dec. 10, 2009

(54) USE OF BIARYLCARBOXAMIES IN THE TREATMENT OF HEDGEHOG PATHWAY-RELATED DISORDERS

(76) Inventors: Rishi Kumar Jain, Cambridge, MA (US); Joseph Kelleher, Somerville, MA (US); Stefan Peukert, Arlington, MA (US); Yingchuan Sun, Arlington, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/297,158

(22) PCT Filed: Apr. 12, 2007
(86) PCT No.: PCT/US07/09138

§ 371 Date: Oct. 14, 2008

Related U.S. Application Data
(60) Provisional application No. 60/792,162, filed on Apr. 14, 2006.

(51) Int. Cl. A61K 31/44; A61K 31/165; A61K 31/42; A61K 31/425

(52) U.S. Cl. 514/352; 514/617; 514/378; 514/365

(57) ABSTRACT

The invention provides methods for modulating, e.g., antagonizing, the activity of the Hedgehog signaling pathway. In particular, the invention provides methods for inhibiting aberrant growth states resulting from phenotypes such as Ptc loss-of-function, Hedgehog gain-of-function, smoothened gain-of-function or Gli gain-of-function, comprising contacting a cell with a sufficient amount of a compound of the invention (e.g., a compound of Formula I).

### BACKGROUND OF THE INVENTION

Hedgehog (Hh) signaling was first identified in Drosophila as an important regulatory mechanism for embryonic pattern formation, or the process by which embryonic cells form ordered spatial arrangements of differentiated tissues. (Nusslein-Volhard et al. (1980) Nature 287, 795-801) In mammalian cells, three Hedgehog genes, Sonic Hedgehog (Shh), India Hedgehog (Ihh) and Desert Hedgehog (Dhh), have been identified. Hedgehog genes encode secreted proteins, which undergo post-translational modifications, including autocatalytic cleavage and lipid modification (palmitoylation) at the N-terminus and cholesterol modification of the C-terminus.

The lipid-modified N-terminal Hedgehog protein triggers the signaling activity of the protein pathway, and cell to cell communication is engendered by the dispatch of soluble Hedgehog protein from a signaling cell and receipt by a responding cell. In responding cells, the 12-pass transmembrane receptor Patched (Ptch) acts as negative regulator of Hh signaling and the 7-pass transmembrane protein Smoothened (Smo) acts as a positive regulator of Hh signaling. At resting state, free Ptch (i.e., unbound by Hh) substoichiometrically suppresses pathway activity induced by Smo (Taipale et al. (2002) Nature 418: 892); upon binding ligand Hh protein, however, repression of Smo is relieved, and the resulting signaling cascade leads to the activation and nuclear translocation of Gli transcription factors (Gli1, Gli2 and Gli3).

Downstream target genes of Hh signaling transcription include Wnts, TGFβ, and Ptc and Gli1, which are elements of the positive and negative regulatory feedback loop. Several cell-cycle and proliferation regulatory genes, such as c-myc, cyclin D and E are also among the target genes of Hh signaling.

Hh signaling is known to regulate a diverse range of biological processes, such as cellular proliferation, differentiation, and organ formation in a tissue specific and dose dependent manner. In the development of neural tubes, Shh is expressed in the floorplate and directs the differentiation of specific subtypes of neurons, including motor and dopaminergic neurons. Hh is also known to regulate the proliferation of neuronal progenitor cells, such as cerebella granule cells and neural stem cells. In the developing intestinal tract, a low-level of Hh signaling is required for pancreatic development, while a high-level of Hh signaling blocks pancreatic organogenesis. Hh is also known to play important roles in stem cell proliferation and organogenesis in skin, prostate, testis and bone marrow.

Normally, Hh signaling is strictly controlled during cellular proliferation, differentiation and embryonic pattern formation. However, aberrant activity of the Hedgehog signaling pathway, due to mutations that constitutively activate the pathway, for instance, may have pathological consequences. By way of example, loss-of-function mutations of Patched are found in Gorlin's syndrome (a hereditary syndrome with high risk of skin and brain cancers, also known as Basal Cell Nevus Syndrome (BCNS)); and gain-of-function mutations of Smo and Gli are linked to basal cell carcinoma and glioblastoma. Basal cell carcinoma (BCC) is the most common form of skin cancer, affecting more than 90,000 Americans each year. Constitutive activation of Hh has been found to promote tumorigenesis in BCC, medulloblastoma (the most common childhood brain tumor), rhabdomyosarcoma, pancreatic cancer, small cell lung cancer, prostate cancer and breast cancer. Besides the roles in tumorigenesis, Hh signaling is also implicated in the metastasis of prostate cancer. Hh signaling may be involved in many additional types of tumor types and such links are expected to continue to be discovered; this is an area of active research in many cancer centers around the world.

Proliferation of these cancer cells requires Hh activation, and blocking Hh signaling pathways often inhibits cancer cell proliferation. Indeed, Hh antagonist cyclopamine and Gli1 siRNA can effectively block the proliferation of these cancer cells, and can reduce tumor size in Xenograft models, suggesting that novel Hh antagonists could provide new chemotherapeutic agents for the treatment of these cancers. Hh antagonist cyclopamine has been shown to suppress the metastasis of prostate cancer in animal models.

In addition to being involved in cancer, Hh signaling plays important roles in normal tissue homeostasis and regeneration. Hh pathway is activated after the injury of retina, bile duct, lung, bone and prostate in mouse models. Hh pathway is also constantly active in hair follicles, bone marrow, and certain regions of the central nervous system (CNS), and benign prostate hyperplasia and blood vessel formation in wet macular degeneration require Hedgehog pathway activity. Cellular regeneration processes can be blocked by anti-Shh antibody and cyclopamine. Therefore, small molecule antagonists of Hh signaling pathway might be useful in the treatment of neuronal proliferative diseases, benign prostate hyperplasia, wet macular degeneration, psoriasis, bone marrow proliferative diseases and leukemias, osteopetrosis and hair removal.

Evidence that constitutive activation of Smo results in cancers (e.g., BCC), and that Smo may be oncogenic upon its release from inhibition by Ptch, suggests utility of Smo antagonists as therapeutic agents in the treatment of such disorders. (Stone et al. (1996) Nature 384: 129). Accordingly, molecules that modulate the activity of the Hedgehog signaling pathway, e.g., which modulate Smo activity, are therapeutically useful.

### SUMMARY OF THE INVENTION

The present invention relates generally to the diagnosis and treatment of pathologies relating to the Hedgehog pathway, including but not limited to tumor formation, cancer, neoplasia, and non-malignant hyperproliferative disorders, and more particularly to methods of inhibiting tumorigenesis, tumor growth and tumor survival using agents that inhibit the Hedgehog and Smo signaling pathway, e.g., the compounds of the invention (e.g., a compound of Formula I (e.g., of Formulae (Ia), (Ib) or (Ic)). The methods and compounds of the present invention relate to inhibiting activation of the Hedgehog signaling pathway, e.g., by inhibiting aberrant growth states resulting from phenotypes such as Ptc loss-of-function, Hedgehog gain-of-function, Smoothened gain-of-function or Gli gain-of-function, and comprise contacting the cell with a compounds of the invention (e.g., a compound of Formula I) in a sufficient amount to agonize a normal Ptc activity, antagonize a normal Hedgehog activity, or antagonize smoothened activity (e.g., to reverse or control the aberrant growth state).

The compounds of the invention, as further described below, include small molecule inhibitors or antagonists of Smo synthesis, expression, production, stabilization, phosphorylation, relocation within the cell, and/or activity. The compounds of the invention include but are not limited to compounds of Formula I.

One aspect of the present invention makes available methods employing compounds for inhibiting Smo-dependent pathway activation. Another aspect of the present invention makes available methods employing compounds for inhibiting Hedgehog (ligand)-independent pathway activation. In certain embodiments, the present methods can be used to counteract the phenotypic effects of unwanted activation of a Hedgehog pathway, such as resulting from Hedgehog gain-of-function, Ptc loss-of-function or smoothened gain-of-function mutations. For instance, the subject method can involve contacting a cell (in vitro or in vivo) with a Smo antagonist, such as a compound of the invention (e.g., a compound of Formula I) or other small molecule in an amount sufficient to antagonize a smoothened-dependent and/or Hedgehog independent activation pathway.

The methods of the present invention may be used to regulate proliferation and/or differentiation of cells in vitro and/or in vivo, e.g., in the formation of tissue from stem cells, or to prevent the growth of hyperproliferative cells. In another particular embodiment, contacting the cell with—or introducing into the cell—a compound of the invention (e.g., a compound of Formula I) results in inhibition of cellular proliferation, inhibition of tumor cell growth and/or survival, and/or inhibition of tumorigenesis. Thus, another particular embodiment provides methods for inhibiting and/or antagonizing the Hh pathway by employing compounds of the invention (e.g., a compound of Formula I) in a tumor cell.

The methods of the present invention may employ compounds of the invention (e.g., a compound of Formula I) as formulated as pharmaceutical preparations comprising a pharmaceutically acceptable excipient or carrier, and said preparations may be administered to a patient to treat conditions involving unwanted cell proliferation such as cancers and/or tumors (such as medullablastoma, basal cell carcinoma, etc.), and non-malignant hyperproliferative disorders.

One embodiment of the present invention provides a method for inhibiting the synthesis, expression, production, stabilization, phosphorylation, relocation within the cell, and/or activity of a Smo protein in a cell in vitro or in vivo comprising, contacting said cell with, or introducing into said cell, a compound of the invention (e.g., a compound of Formula I).

Another aspect of the invention provides a method of diagnosing, preventing and/or treating cellular debilitations, derangements, and/or dysfunctions; hyperplastic, hyperproliferative and/or cancerous disease states; and/or metastasis of tumor cells, in a mammal characterized by the presence and/or expression of a Smo gene or gene product (e.g., a Smo protein), comprising administering to a mammal a therapeutically effective amount of a compound of the invention (e.g., a compound of Formula I).

Yet another aspect of the invention provides a method of treating apoptotic resistant tumor cells comprising administering a compound of the invention (e.g., a compound of Formula I) to said tumor cell in vitro or in vivo. In one embodiment, the method comprises the use of a compound of the invention (e.g., a compound of Formula I) as a means of inducing a tumor cell to undergo senescence, apoptosis, or necrosis. In another embodiment, said administering results in tumor cell death and prevention from metastasis.

Another aspect of the invention provides a method of overcoming resistance to chemotherapeutic agents in tumor cells, comprising administering compound of the invention (e.g., a compound of Formula I) to the cell, wherein said administering results in increased sensitivity of the tumor cell to said chemotherapeutic agent and results in subsequent tumor cell death and prevention from metastasis.

### BRIEF DESCRIPTION OF THE FIGURES

FIG. 1a shows a general synthetic scheme for the preparation of compounds of Formula I. Most preferred compounds of Formula (Ic) can be prepared by reductive amination from intermediate 5a with aldehydes R6(CH2)nCHO in the presence of a reducing agent such as sodium triacetoxy borohydride as shown in FIG. 1b.

### DETAILED DESCRIPTION OF THE INVENTION

The present invention relates to compounds of the invention, including biarylcarboxamide compounds, of the formula (I):

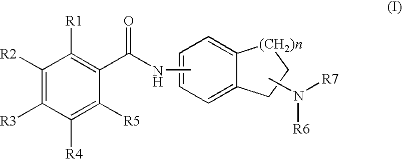

wherein

R2-C, R3-C, R4-C or R5-C may be replaced by N

n is 1, 2 or 3

R1 is carbocyclic aryl or heteroaryl

R2, R3, R4 and R5 are independently hydrogen, lower alkyl, lower alkoxy, lower alkylthio, fluoro, chloro, bromo, amino, substituted amino, trifluoromethyl, acyloxy, alkylcarbonyl, trifluoromethoxy or cyano

R6 is hydrogen, optionally substituted alkyl, carbocyclic or heterocyclic aryl-lower alkyl

R7 is hydrogen, optionally substituted alkyl, carbocyclic aryl, heteroaryl, carbocyclic aryl-lower alkyl, heteroaryl-lower alkyl, or

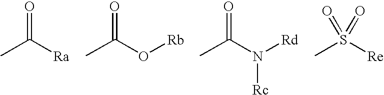

wherein

Ra is optionally substituted alkyl, cycloalkyl, aryl or heterocyclyl

Rb is optionally substituted alkyl, cycloalkyl, aryl or heterocyclyl

Re and Rd are independently hydrogen, substituted alkyl, cycloalkyl, aryl; or

heterocyclyl, or Rc and Rd together represent lower alkylene or lower alkylene interrupted by O, S, N—(H, alkyl, arylalkyl);

Re is optionally substituted alkyl, cycloalkyl, aryl or heterocyclyl, amino or substituted amino

and pharmaceutically acceptable salts thereof, and enantiomers thereof.

A preferred embodiment of the invention relates to compounds of Formula (Ia)

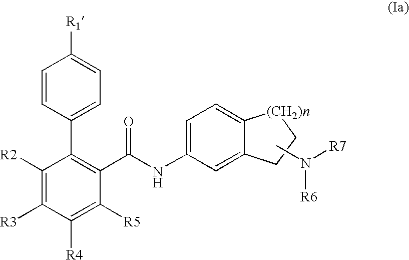

wherein R2-C, R3-C, R4-C or R5-C may be replaced by N

wherein

R1′ is hydrogen, fluoro, chloro, bromo, lower alkyl, cyano, methoxy, trifluoromethyl, trifluoromethoxy, dimethylamino

R2 to R7 have meaning as defined for Formula I,

and pharmaceutically acceptable salts thereof, and enantiomers thereof.

Another preferred embodiment of the invention relates to compounds of Formula (Ib)

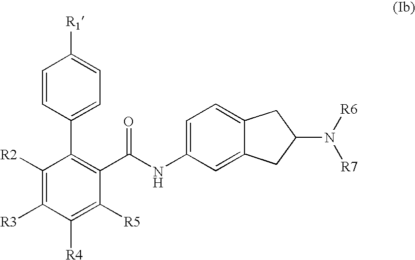

wherein

R1′ is trifluoromethyl, chloro, fluoro

R2 and R3 are independently hydrogen, C1-C4 alkyl, C1-C4-alkoxy, trifluoromethyl, chloro or fluoro

R4 and R5 are hydrogen

R6 is hydrogen or C1-C3 alkyl

R7 is optionally substituted alkyl, carbocyclic aryl, heteroaryl, carbocyclic aryl-lower alkyl, heteroaryl-lower alkyl, or

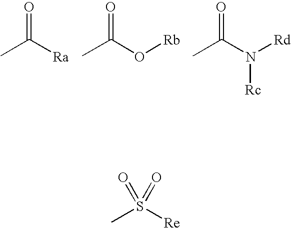

wherein

Ra is optionally substituted alkyl, cycloalkyl, aryl or heterocyclyl

Rb is optionally substituted alkyl, cycloalkyl, aryl or heterocyclyl

Rc and Rd are independently hydrogen, substituted alkyl, cycloalkyl, aryl; or

heterocyclyl, or Rc and Rd together represent lower alkylene or lower alkylene interrupted by O, S, N—(H, alkyl, arylalkyl)

Re is optionally substituted alkyl cycloalkyl, aryl or heterocyclyl, amino or substituted amino

and pharmaceutically acceptable salts thereof, and enantiomers thereof.

Another preferred embodiment of the invention relates to compounds of Formula (Ib)

wherein

R1′ is trifluoromethyl, chloro, fluoro

R2 and R3 are independently hydrogen, C1-C4 alkyl, C1-C4-alkoxy, trifluoromethyl, chloro or fluoro

R4 and R5 are hydrogen

R6 is hydrogen

R7 is optionally substituted alkyl, carbocyclic aryl, heteroaryl, carbocyclic aryl-lower alkyl or heteroaryl-lower alkyl

and pharmaceutically acceptable salts thereof, and enantiomers thereof.

Another particularly preferred embodiment of the invention relates to compounds of Formula (Ic)

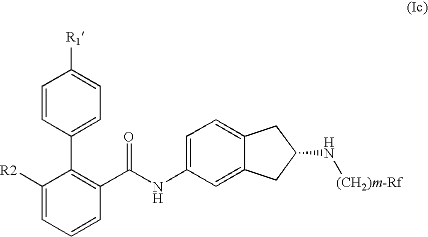

wherein

R1′ is trifluoromethyl or chloro

R2 is hydrogen or methyl

m is 0 or 1;

Rf is carbocyclic or heterocyclic aryl

and pharmaceutically acceptable salts thereof.

The compounds of the invention, depending on the nature of the substituents described herein, possess one or more asymmetric carbon atoms, and therefore exist as racemates, and the R and S enantiomer thereof. Preferred is the more active enantiomer, typically assigned the S configuration (at the carbon with the NR6R7 substituent).

As further described below, the compounds of the invention can be prepared as described in PCT patent publications WO01/05767 and WO00/05201, and in Ksander, et al. (2001) Journal of Medicinal Chemistry, 44:4677, the contents of all of which are herein incorporated by reference.

In the present description, the term “treatment” includes both prophylactic or preventive treatment as well as curative or disease suppressive treatment, including treatment of patients at risk for a disorder of the invention (e.g., a Hedgehog-related disorder (e.g., cancer)) as well as ill patients. This term further includes the treatment for the delay of progression of the disease.

By “suppress and/or reverse,” e.g., a Hedgehog-related disorder (e.g., cancer), Applicants mean to abrogate said Hedgehog-related disorder (e.g., diabetes), or to render said condition less severe than before or without the treatment.

“Cure” as used herein means to lead to the remission of the Hedgehog-related disorder (e.g., cancer) in a patient, or of ongoing episodes thereof, through treatment.

The terms “prophylaxis” or “prevention” means impeding the onset or recurrence of metabolic disorders, e.g., diabetes.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted.

“Diagnosis” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

“Subject” or “patient” refers to a mammal, preferably a human, in need of treatment for a condition, disorder or disease.

“A compound(s) of the invention” as used herein includes but is not limited to compounds of Formula I (e.g., a compound of Formulae (Ia), (Ib) or (Ic)). A compound of the invention includes the specifically listed compounds listed herein, including those listed in the Examples of the present application.

“Delay of progression” as used herein means that the administration of a compound of the invention (e.g., a compound of Formula I) to patients in a pre-stage or in an early phase of a Hedgehog-related disorder (e.g., cancer) prevents the disease from evolving further, or slows down the evolution of the disease in comparison to the evolution of the disease without administration of the active compound.

“Hedgehog gain-of-function” refers to an aberrant modification or mutation of a Ptc gene, Hedgehog gene, or smoothened gene, or a change (e.g., decrease) in the level of expression of such a gene, which results in a phenotype which resembles contacting a cell with a Hedgehog protein, e.g., aberrant activation of a Hedgehog pathway. The gain-of-function may include a loss of the ability of the Ptc gene product to regulate the level of expression of Gli genes, e.g., Gli1, Gli2, and Gli3, or loss of the ability to regulate the processing, stability, localization or activity of the Gli proteins, e.g., Gli1, Gli2, and Gli3. The term “Hedgehog gain-of-function” is also used herein to refer to any similar cellular phenotype (e.g., exhibiting excess proliferation) which occurs due to an alteration anywhere in the Hedgehog signal transduction pathway, including, but not limited to, a modification or mutation of Hedgehog itself. For example, a tumor cell with an abnormally high proliferation rate due to activation of the Hedgehog signaling pathway would have a “Hedgehog gain-of-function” phenotype, even if Hedgehog is not mutated in that cell.

“Patched loss-of-function” refers to an aberrant modification or mutation of a Ptc gene, or a decreased level of expression of the gene, which results in a phenotype which resembles contacting a cell with a Hedgehog protein, e.g., aberrant activation of a Hedgehog pathway. The loss-of-function may include a loss of the ability of the Ptc gene product to regulate the level of expression, processing, stability, localization, regulation or activity of Gli genes and proteins, e.g., Gli 1, Gli2 and Gli3.

“Gli gain-of-function” refers to an aberrant modification or mutation of a Gli gene, or an increased level of expression of the gene, which results in a phenotype which resembles contacting a cell with a Hedgehog protein, e.g., aberrant activation of a Hedgehog pathway.

“Smoothened gain-of-function” refers to an aberrant modification or mutation of a Smo gene, or an increased level of expression of the gene, which results in a phenotype which resembles contacting a cell with a Hedgehog protein, e.g., aberrant activation of a Hedgehog pathway.

As used herein a “small organic molecule” is an organic compound (or organic compound complexed with an inorganic compound (e.g., metal)) that has a molecular weight of less than 3 kilodaltons, and preferably less than 1.5 kilodaltons.

As used herein a “reporter” gene is used interchangeably with the term “marker gene” and is a nucleic acid that is readily detectable and/or encodes a gene product that is readily detectable such as luciferase.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Agent” refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Analog” as used herein, refers to a small organic compound, a nucleotide, a protein, or a polypeptide that possesses similar or identical activity or function(s) as the compound, nucleotide, protein or polypeptide or compound having the desired activity and therapeutic effect of the present invention. (e.g., inhibition of tumor growth), but need not necessarily comprise a sequence or structure that is similar or identical to the sequence or structure of the preferred embodiment

“Apoptosis” refers to programmed cell death and is characterized by certain cellular characteristics such as membrane blebbing, chromatin condensation and fragmentation, formation of apoptotic bodies and a positive “TUNEL” staining pattern. Degradation of genomic DNA during apoptosis results in formation of characteristic, nucleosome sized DNA fragments; his degradation produces a diagnostic (about) 180 bp laddering pattern when analyzed by gel electrophoresis. A later step in the apoptotic process is degradation of the plasma membrane, rendering apoptotic cells leaky to various dyes (e.g., trypan blue and propidium iodide).

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

“Inhibitors,” or “antagonists” refer to inhibitory molecules identified using in vitro and in vivo assays for Hh pathway function, e.g., Smo antagonists. In particular, inhibitors and antagonists refer to compounds or agents that decrease signaling that occurs via the Hh pathway. Inhibitors may be compounds that decrease, block, or prevent, signaling via this pathway.

“Hedgehog-related disorder(s)” as used herein includes disorders associated with disruption or aberrance of the Hedgehog pathway, as well as disorders associated with normal but undesired growth states relating to activation of the Hedgehog pathway. “Hedgehog-related disorder(s)” include but are not limited to tumor formation, cancer, neoplasia, malignant hyperproliferative disorders, and non-malignant hyperproliferative disorders. “Hedgehog-related disorder(s)” also include benign prostate hyperplasia, psoriasis, wet macular degeneration, osteopetrosis and unwanted hair growth.

As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin, central nervous system including brain; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc. Both human and veterinary uses are contemplated.

Cancers which are particularly amenable to treatment by the methods of the invention include but are not limited to gliomas, medulloblastomas, primitive neuroectodermal tumors (PNETS), basal cell carcinoma (BCC), small cell lung cancers, large cell lung cancers, tumors of the gastrointestinal tract, rhabdomyosarcomas, soft tissue sarcomas, pancreatic tumors, bladder tumors and prostate tumors.

As used herein, the term “malignant hyperproliferative disorder(s)” includes but is not limited to cancers, neuronal proliferative disorders, bone marrow proliferative diseases and leukemias.

As used herein, the term “non-malignant hyperproliferative disorder(s)” includes but is not limited to non-malignant and non-neoplastic proliferative disorders, such as smooth muscle hyperplasia in blood vessels, cutaneous scarring, and pulmonary fibrosis.

The term “alkyl” refers to straight or branched chain hydrocarbon groups having 1 to 20 carbon atoms, preferably lower alkyl of 1 to 7 carbon atoms. Exemplary alkyl groups include methyl, ethyl, propyl, isopropyl, n-butyl, t-butyl, isobutyl, pentyl, hexyl, isohexyl, heptyl, 4,4-dimethylpentyl, octyl and the like. Preferred is C₁-C₄-alkyl.

The term “lower” referred to herein in connection with organic radicals or compounds respectively generally defines, if not defined differently, such with up to and including 7, preferably up and including 4 and advantageously one or two carbon atoms. Such may be straight chain or branched.

The term “optionally substituted alkyl” refers to unsubstituted or substituted straight or branched chain hydrocarbon groups having 1 to 20 carbon atoms, preferably lower alkyl of 1 to 7 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like.

The term “substituted alkyl” refers to alkyl groups substituted by one or more of the following groups: halo (such as F, Cl, Br and I), hydroxy, alkoxy, alkoxyalkoxy, aryloxy, cycloalkyl, alkanoyl, alkanoyloxy, amino, substituted amino, alkanoylamino, thiol, alkylthio, arylthio, alkylthiono, alkylsulfonyl, arylsulfonyl, heteroarylsulfonyl, aminosulfonyl, nitro, cyano, carboxy, carbamyl, alkoxycarbonyl, aryl, aralkoxy, guanidino, heterocyclyl (e.g., indolyl, imidazolyl, furyl, thienyl, thiazolyl, pyrrolidyl, pyridyl, pyrimidyl), and the like.

The term “lower alkyl” refers to those alkyl groups as described above having 1 to 7, preferably 1 to 4 carbon atoms. The term “halogen” or “halo” refers to fluorine, chlorine, bromine and iodine.

The term “alkoxy” or “alkyloxy” refers to alkyl-O—.

The term “aryl” or “ar”, refers to carbocyclic monocyclic or bicyclic aromatic hydrocarbon groups having 6 to 12 carbon atoms in the ring portion, such as phenyl, naphthyl, tetrahydronaphthyl, and biphenyl groups, each of which may optionally be substituted by one to four, e.g., one or two, substituents such as alkyl, halo, trifluoromethyl, hydroxy, alkoxy, alkanoyl, alkanoyloxy, amino, substituted amino, alkanoylamino, thiol, alkylthio, nitro, cyano, carboxy, carboxyalkyl, carbamyl, alkoxycarbonyl, alkylthiono, alkylsulfonyl, aminosulfonyl, and the like.

The term “aralkyl” refers to an aryl group linked to an alkyl group, such as benzyl.

The term “halogen” or “halo’ refers to fluorine, chlorine, bromine and iodine.

The term “haloalkyl” refers to alkyl which mono- or polysubstituted by halo, such as trifluoromethoxy.

The term “alkylene” refers to a straight chain bridge of 1 to 6 carbon atoms connected by single bonds (e.g., —(CH2)ₓ- wherein x is 1 to 6) which may be substituted with 1 to 3 lower alkyl groups.

The term “alkylene interrupted by O, S, N—(H, alkyl or aralkyl)” refers to a straight chain of 2 to 6 carbon atoms which is interrupted by O, S, N—(H, alkyl or aralkyl), such as (m)ethyleneoxy(m)ethylene, (m)ethylenethio(m)ethylene, or (m)ethyleneimino(m)ethylene.

The term “cycloalkyl” refers to cyclic hydrocarbon groups of 3 to 8 carbon atoms such as cyclopentyl, cyclohexyl or cycloheptyl.

The term “alkanoyloxy refers to alkyl-C(O)—O—

The terms “alkylamino” and “dialkylamino” refer to (alkyl)NH— and (alkyl)₂N—, respectively.

The term “alkanoylamino” refers to alkyl-C(O)—NH—.

The term “alkylthio” refers to alkyl-S—.

The term “alkylthiono” refers to alkyl-S(O)—.

The term “alkylsulfonyl” refers to alkyl-S(O)₂—

The term “carbamyl” refers to —C(O)-amino or —C(O)-substituted amino.

The term “alkoxycarbonyl”, refers to alkyl-O—C(O)—.

The term “acyl” refers to alkanoyl, aroyl, heteroaryol, aryl-alkanoyl, heteroarylalkanoyl, and the like.

The term “heteroaryl” or “heteroar” refers to an aromatic heterocycle, for example monocyclic or bicyclic heterocyclic aryl, such as pyrrolyl, pyrazolyl, imidazolyl, oxazolyl, thiazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl, and the like, optionally substituted by one to four, e.g. one or two, substituents, such as lower alkyl, lower alkoxy or halo, the point of attachment of said heterocycle being at a carbon atom of the heterocyclic ring. Preferred heteroaryl residues are 1-methyl-2-pyrrolyl, 2-,3-thienyl, 2-thiazolyl, 2-imidazolyl, 1-methyl-2-imidazolyl, 2-,3-,4-pyridyl, or 2-quinolyl.

The term “alkanoyl” refers, for example, to C₂-C₇-alkanoyl, especially C₂-C₅-alkanoyl, such as acetyl, propionyl or pivaloyl.

The term “aralkoxy” refers to an aryl group linked to an alkoxy group.

The term “arylsulfonyl” refers to aryl-SO₂—.

The term “aroyl” refers to aryl-CO—.

The term “heterocyclyl” refers to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazoliclinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like.

Exemplary bicyclic heterocyclic groups include indolyl, benzothiazolyl, benzoxazolyl, benzothienyl, quinuclidinyl, quinolinyl, tetrahydroisoquinolinyl, isoquinolinyl, benzimidazolyl, benzopyranyl, indolizinyl, benzofuryl, chromonyl, coumarinyl, enzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl) and the like.

Exemplary tricyclic heterocyclic groups include carbazolyl, benzidolyl, phenanthrolinyl, acridinyl, phenanthridinyl, xanthenyl and the like.

The term “heterocyclyl” also includes substituted heterocyclic groups. Substituted heterocyclic groups refer to heterocyclic groups substituted with 1, 2 or 3 of the following:

(a) alkyl

(b) hydroxy (or protected hydroxy)

(c) halo;

(d) oxo (i.e. ═O)

(e) amino or substituted amino

(f) alkoxy

(g) cycloalkyl

(h) carboxy

(i) heterocyclooxy

(j) alkoxycarbonyl, such as unsubstituted lower alkoxycarbonyl

(k) carbamyl, alkylcarbamyl, arylcarbamyl, dialkylcarbamyl

(l) mercapto

(m) nitro

(n) cyano

(o) sulfonamido, sulfonamidoalkyl or sulfonamidodialkyl

(p) aryl

(q) alkylcarbonyloxy

(r) arylcarbonyloxy,

(s) arylthio

(t) aryloxy

(u) alkylthio

(v) formyl

(w) arylalkyl; or

(x) aryl substituted with alkyl, cycloalkyl, alkoxy, hydroxy, amino, alkylamino, dialkylamino or halo.

The term “heterocyclooxy” denotes a heterocyclic group bonded through an oxygen bridge.

The term “heteroaryl” or “heteroar” refers to an aromatic heterocycle, for example monocyclic or bicyclic aryl, such as pyrrolyl, pyrazolyl, imidazolyl, oxazolyl, isoxazolyl, thiazolyl, isothiazolyl, furyl, thienyl, pyrazinyl, pyrimidinyl, pyridazinyl, indolyl; benzothiazolyl, benzoxazolyl, benzothienyl, quinolinyl, isoquinolinyl, benzimidazolyl, benzofuryl, and the like, optionally substituted by e.g., lower alkyl, lower alkoxy or halo.

The term “heteroarylsulfonyl” refers to heteroaryl-SO₂—

The term “heteroaroyl” refers to heteroaryl-CO—.

The term “acylamino” refer to acyl-NH—.

The term “substituted amino” refers to amino mono- or, independently, disubstituted by alkyl, aralkyl, aryl, heteroaryl, cycloalkyl, cycloalkylalkyl, heteroaralkyl, or disubstituted by lower alkylene or lower alkylene interrupted by O, S, N—(H, alkyl, aralkyl) and the like.

Pharmaceutically acceptable salts of any acidic compounds of the invention are salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris-(hydroxymethyl)-methylammonium salts.

Similarly acid addition salts, such as of mineral acids, organic carboxylic, and organic sulfonic acids e.g., hydrochloric acid, methanesulfonic acid, maleic acid, are possible provided a basic group, such as amino or pyridyl, constitutes part of the structure.

Pharmaceutically acceptable salts of the compounds of the invention are particularly acid addition salts, such as of mineral acids, organic carboxylic, and organic sulfonic acids e.g., hydrochloric acid, methanesulfonic acid, maleic acid, and the like provided a basic group, such as pyridyl, constitutes part of the structure.

The compounds of the invention depending on the nature of the substituents, possess one or more asymmetric carbon atoms, and therefore exist as racernates and the (R) and (S) enantiomers thereof. All are within the scope of the invention. Preferred is the more active enantiomer typically assigned the S-configuration (at the carbon being the NR₆R₇ substituent).

The present invention relates to the discovery that signal transduction pathways regulated by Hh and/or Smo can be modulated by the compounds of the invention (e.g., a compound of Formula I (e.g., of Formulae (Ia), (Ib) or (Ic)).

In one embodiment, the methods of the present invention employ the compounds of the invention (e.g., compounds of Formula I) for inhibiting Smo-dependent pathway activation. Another aspect of the present invention makes available methods employing compounds for inhibiting Hedgehog (ligand)-independent pathway activation. In certain embodiments, the present methods can be used to counteract the phenotypic effects of unwanted activation of a Hedgehog pathway, such as resulting from Hedgehog gain-of-function, Ptc loss-of-function or smoothened gain-of-function mutations. For instance, the subject method can involve contacting a cell (in vitro or in vivo) with a Smo antagonist, such as a compound of the invention (e.g., a compound of Formula I) or other small molecule in an amount sufficient to antagonize a smoothened-dependent and/or Hedgehog independent activation pathway.

In one embodiment, the compounds of the invention (e.g., compounds of Formula I) inhibit Hh signaling by locking the three dimensional structure of the Smo protein in an inactive conformation or preventing Smo from adopting an active conformation. In another embodiment, the compounds of the invention (e.g., compounds of Formula I) inhibit Hh signaling by preventing endogenous activating ligands for Smo from binding to or activating Smo (i.e., acting via negative cooperativity with endogenous agonists). In another embodiment, the compounds of the invention (e.g., compounds of Formula I) inhibit Hh signaling by increasing binding of endogenous inactivating ligands for Smo from binding to or inactivating Smo (i.e., acting via positive cooperativity with endogenous antagonist).

In another embodiment, the compounds of the invention (e.g., compounds of Formula I) inhibit Hh signaling by preventing Smo from localizing to the plasma membrane. In another embodiment, the compounds of the invention (e.g., compounds of Formula I) inhibit Hh signaling by preventing signaling from Ptch to Smo, in the presence or absence of Hh ligand. In another embodiment, the compounds of the invention (e.g., compounds of Formula I) inhibit Hh signaling by preventing the stabilization of Smo. In another embodiment, the compounds of the invention (e.g., compounds of Formula I) inhibit Hh signaling by preventing the phosphorylation of Smo on activating sites. In another embodiment, the compounds of the invention (e.g., compounds of Formula I) inhibit Hh signaling by increasing the phosphorylation of Smo on inhibitory sites.

In still another embodiment, the compounds of the invention (e.g., compounds of Formula I) inhibit Hh signaling by preventing Smo from activating downstream targets, such as transcription factor Gli. In another embodiment, the compounds of the invention (e.g., compounds of Formula I) inhibit Hh signaling by effecting the inactivation, sequestration, and/or degradation of Smo.

In another embodiment, the methods of the present invention may be used to regulate proliferation and/or differentiation of cells in vitro and/or in vivo, e.g., in the formation of tissue from stem cells, or to prevent the growth of hyperproliferative cells. In another particular embodiment, contacting the cell with—or introducing into the cell—a compound of the invention (e.g., a compound of Formula I) results in inhibition of cellular proliferation, inhibition of cancer/tumor cell growth and/or survival, and/or inhibition of tumorigenesis. Thus, another particular embodiment provides methods for inhibition and/or antagonism of the Hh pathway by employing compounds of the invention (e.g., a compound of Formula I) in a tumor cell.

In yet another embodiment, the methods of the present invention employ compounds of the invention (e.g., a compound of Formula I) as formulated as a pharmaceutical preparation comprising a pharmaceutically acceptable excipient or carrier, and said preparations may be administered to a patient to treat conditions involving unwanted cell proliferation such as cancers and/or tumors (such as medullablastoma, basal cell carcinoma, etc.), and non-malignant hyperproliferative disorders.

One embodiment of the present invention provides a method for inhibiting the synthesis, expression, production, and/or activity of a Smo protein in a cell in vitro or in vivo comprising, contacting said cell with, or introducing into said cell, a compound of the invention (e.g., a compound of Formula I).

Another embodiment of the invention provides a method of diagnosing, preventing and/or treating cellular debilitations, derangements, and/or dysfunctions; hyperplastic, hyperproliferative and/or cancerous disease states; and/or metastasis of tumor cells, in a mammal characterized by the presence and/or expression of a Smo gene or gene product (e.g., a Smo protein), comprising administering to a mammal a therapeutically effective amount of an agent that inhibits or antagonizes the synthesis and/or expression and/or activity of a compound of the invention (e.g., a compound of Formula I).

In yet another embodiment, the invention provides a method of treating apoptotic resistant tumor cells comprising administering a compound of the invention (e.g., a compound of Formula I) to said tumor cell in vitro or in vivo. In one embodiment, the method comprises the use of a compound of the invention (e.g., a compound of Formula I) as a means of inducing a tumor cell to undergo senescence, apoptosis, or necrosis. In another embodiment, said administering results in tumor cell death and prevention from metastasis.

Another embodiment of the invention provides a method of overcoming resistance to chemotherapeutic agents in tumor cells, comprising administering compound of the invention (e.g., a compound of Formula I) to the cell, wherein said administering results in increased sensitivity of the tumor cell to said chemotherapeutic agent and results in subsequent tumor cell death and prevention from metastasis.

It is, therefore, specifically contemplated that compounds of Formula I which interfere with aspects of Hh, Ptc, or smoothened signal transduction activity will likewise be capable of inhibiting proliferation (or other biological consequences) in normal cells and/or cells having a patched loss-of-function phenotype, a Hedgehog gain-of-function phenotype, a smoothened gain-of-function phenotype or a Gli gain-of-function phenotype. Thus, it is contemplated that in certain embodiments, these compounds may be useful for inhibiting Hedgehog activity in normal cells, e.g., which do not have a genetic mutation that activates the Hedgehog pathway. In preferred embodiments, the compounds are capable of inhibiting at least some of the biological activities of Hedgehog proteins, preferably specifically in target cells.

Thus, the methods of the present invention include the use of compounds of Formula I which agonize Ptc inhibition of Hedgehog signaling, such as by inhibiting activation of smoothened or downstream components of the signal pathway, in the regulation of repair and/or functional performance of a wide range of cells, tissues and organs, including normal cells, tissues, and organs, as well as those having the phenotype of Ptc loss-of-function, Hedgehog gain-of-function, smoothened gain-of-function or Gli gain-of-function. For instance, the subject method has therapeutic and cosmetic applications ranging from regulation of neural tissues, bone and cartilage formation and repair, regulation of spermatogenesis, regulation of benign prostate hyperplasia, regulation of blood vessel formation in wet macular degeneration, psoriasis, regulation of smooth muscle, regulation of lung, liver and other organs arising from the primitive gut, regulation of hematopoietic function, regulation of skin and hair growth, etc. Moreover, the subject methods can be performed on cells which are provided in culture (in vitro), or on cells in a whole animal (in vivo).

In certain embodiments, a compound of Formula I can inhibit activation of a Hedgehog pathway by binding to smoothened or its downstream proteins.

In another embodiment, the present invention provides the use of pharmaceutical preparations comprising, as an active ingredient, a Hedgehog signaling modulator such as a compound of Formula I, a smoothened antagonist such as described herein, formulated in an amount sufficient to inhibit, in vivo, proliferation or other biological consequences of Ptc loss-of-function, Hedgehog gain-of-function, smoothened gain-of-function or Gli gain-of-function.

The treatment of subjects by administering compounds of the invention (e.g., compounds of Formula I) can be effective for both human and animal subjects. Animal subjects to which the invention is applicable extend to both domestic animals and livestock, raised either as pets or for commercial purposes. Examples are dogs, cats, cattle, horses, sheep, hogs, goats, and llamas.

The present invention also makes available methods and compounds for inhibiting activation of the Hedgehog signaling pathway, e.g., to inhibit normal but undesired growth states, for example benign prostate hyperplasia or blood vessel formation in wet macular degeneration, resulting from physiological activation of the Hedgehog signaling pathway, comprising contacting the cell with a compound of Formula I, in a sufficient amount to antagonize smoothened activity, or antagonize Gli activity, e.g., to reverse or control the normal growth state.

The present invention makes available methods and compounds for inhibiting activation of the Hedgehog signaling pathway, e.g., to inhibit aberrant growth states resulting from phenotypes such as Ptc loss-of-function, Hedgehog gain-of-function, smoothened gain-of-function or Gli gain-of-function, comprising contacting the cell with a compound of Formula I, in a sufficient amount to antagonize smoothened activity, or antagonize Gli activity e.g., to reverse or control the aberrant growth state.

Members of the Hedgehog family of signaling molecules mediate many important short- and long-range patterning processes during vertebrate development. Pattern formation is the activity by which embryonic cells form ordered spatial arrangements of differentiated tissues. The physical complexity of higher organisms arises during embryogenesis through the interplay of cell-intrinsic lineage and cell-extrinsic signaling. Inductive interactions are essential to embryonic patterning in vertebrate development from the earliest establishment of the body plan, to the patterning of the organ systems, to the generation of diverse cell types during tissue differentiation. The effects of developmental cell interactions are varied: responding cells are diverted from one route of cell differentiation to another by inducing cells that differ from both the uninduced and induced states of the responding cells (inductions). Sometimes cells induce their neighbors to differentiate like themselves (homeogenetic induction); in other cases a cell inhibits its neighbors from differentiating like itself. Cell interactions in early development may be sequential, such that an initial induction between two cell types leads to a progressive amplification of diversity. Moreover, inductive interactions occur not only in embryos, but in adult cells as well, and can act to establish and maintain morphogenetic patterns as well as induce differentiation.

The vertebrate family of Hedgehog genes includes three members that exist in mammals, known as Desert (Dhh), Sonic (Shh) and Indian (Ihh) Hedgehogs, all of which encode secreted proteins. These various Hedgehog proteins consist of a signal peptide, a highly conserved N-terminal region, and a more divergent C-terminal domain. Biochemical studies have shown that autoproteolytic cleavage of the Hh precursor protein proceeds through an internal thioester intermediate which subsequently is cleaved in a nucleophilic substitution. It is likely that the nucleophile is a small lipophilic molecule which becomes covalently bound to the C-terminal end of the N-peptide, tethering it to the cell surface. The biological implications are profound. As a result of the tethering, a high local concentration of N-terminal Hedgehog peptide is generated on the surface of the Hedgehog producing cells. It is this N-terminal peptide which is both necessary and sufficient for short- and long-range Hedgehog signaling activities.

Smoothened (Smo) encodes a 1024 amino acid transmembrane protein that acts as a transducer of the Hedgehog (Hh) signal. Smo protein has 7 hydrophobic membrane-spanning domains, an extracellular amino-terminal region, and an intracellular carboxy-terminal region. Smo bears some similarity to G protein-coupled receptors and is most homologous to the Frizzled (Fz) family of serpentine proteins. (Alcedo et al. (1996) Cell 86: 221)

An inactive Hedgehog signaling pathway is where the transmembrane protein receptor Patched (Ptc) inhibits the stabilization, phosphorylation, and activity of Smoothened (Smo). The transcription factor Gli, a downstream component of Hh signaling, is prevented from entering the nucleus through interactions with cytoplasmic proteins, including Fused (Fu) and Suppressor of fused (Sufu). As a consequence, transcriptional activation of Hedgehog target genes is repressed. Activation of the pathway is initiated through binding of any of the three mammalian ligands (Dhh, Shh or Ihh) to Ptc.

Ligand binding by Hh alters the interaction of Smo and Ptc, reversing the repression of Smo, whereupon Smo moves from internal structures within the cell to the plasma membrane. The localization of Smo to the plasma membrane triggers activation of Hh pathway target genes in an Hh-independent manner. (Zhu et al. (2003) Genes Dev. 17 (10):1240) The cascade activated by Smo leads to the translocation of the active form of the transcription factor Gli to the nucleus. The activation of Smo, through translocated nuclear Gli, activates Hh pathway target gene expression, including of Wnts, TGFβ, and Ptc and Gli themselves.

Increased levels of Hedgehog signaling are sufficient to initiate cancer formation and are required for tumor survival. These cancers include, but are not limited to, prostate cancer (Karhadkar et al. (2004) Nature 431:707; Sanchez et al. (2004) PNAS 101 (34):12561), breast cancer (Kubo et al. (2004) Cancer Res. 64 (17):6071), medulloblastoma (Berman et al. (2002) Science 297 (5586): 1559), basal cell carcinoma (BCC) (Williams et al. (2003) PNAS 100 (8):4616); Xie et al. (1998) Nature 391 (6662):90), pancreatic cancer (Thayer et al. (2003) Nature 425 (6960):851; Berman et al. (2003) Nature 425 (6960):846), small-cell lung cancer (Watkins et al. (2003) Nature 422 (6929):313), glioma (Kinzler et al. (1988) Nature 332:371), cancers of the digestive tract (Berman et al. (2003) Nature 425 (6960):846) and esophageal cancers (Ma et al (2006) Int J Cancer 118 (1):139.

In accordance with the foregoing, the present invention further provides a method for preventing or treating any of the diseases or disorders described above in a subject in need of such treatment, which method comprises administering to said subject a therapeutically effective amount of a compound of the invention (e.g., a compound of Formula I) or a pharmaceutically acceptable salt thereof. For any of the above uses, the required dosage will vary depending on the mode of administration, the particular condition to be treated and the effect desired.

Human patients with Gorlin's syndrome, a hereditary syndrome with high risk of skin and brain cancers, also known as Basal Cell Nevus Syndrome (BCNS) develop basal cell carcinoma (BCC) with high frequency, and other solid tumors (e.g., meduloblastomas) at lower frequency, due to germline loss of function mutations in Ptch. These patients, as well as other, non-Gorlin's patients with BCC who have somatic loss of function mutations in Ptch, are would not be expected to respond to treatments associated with Hedgehog ligands. They would, however, respond to inhibitors of Hh signaling downstream from the Hh ligands, such as the compounds of the invention (e.g., a compound of Formula I), which can act as Smo inhibitors. Similarly, other solid tumors due to patched or Smo mutations will not respond to Hh ligand-related inhibition but will respond to Smo blockade (e.g., by administration of the compounds of the invention).

Administration and Pharmaceutical Compositions:

The invention relates to the use of pharmaceutical compositions comprising compounds of Formula (I), including of Formulae (Ia), (Ib), or (Ic) in the therapeutic (and, in a broader aspect of the invention, prophylactic) treatment of a Hedgehog-related disorder(s).

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with immunomodulatory or anti-inflammatory substances or other anti-tumor therapeutic agents. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

Processes for Making Compounds of the Invention

Representative examples of synthesis of the compounds of the invention, e.g., compounds of Formula (I), including of Formulae (Ia), (Ib) or (Ic) can be found in the Examples section of the present application.

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981.

### EXAMPLES

The present invention is further exemplified, but not limited, by the following representative examples, which are intended to illustrate the invention and are not to be construed as being limitations thereon. The structure of final products described herein can be confirmed by standard analytical methods, e.g., spectrometric and spectroscopic methods (e.g. MS, NMR). Abbreviations used are those conventional in the art. Compounds are purified by standard methods, e.g. crystallization, flash chromatography or reversed phase HPLC.

### Example 1

### Compound Screening

An unbiased cellular pathway-based screen was used to identify compounds that antagonize a Gli-recognition element-luciferase reporter induced by recombinant Shh protein or Smo agonists. Following the initial screen, several filters were applied to identify compounds with desired selectivity. Compounds were identified that may serve both as “lead” compounds for therapeutic development and also as tools to identify possible new components of the Hh signaling pathway, and compounds were divided between (i) antagonists acting on Smo (which, e.g., can be fast-tracked into trials with Gorlin's patients); and (ii) those exerting activity at other points in the Hh pathway. Genomics and proteomics approaches were used in a parallel effort to match newly identified modulators in the pathway with active compounds.

Several reporter cell lines were used to perform primary and secondary screens, including:

TM3 cells (mouse) transfected with a new proprietary reporter construct (pTA-8xGli-Luc). The pTA-8xGli-Luc reporter produces approx. 4 fold more luciferase activity than classical 8xGli-LUC. A highly responsive stable clone has been selected (TMHh12) with >10-fold signal/noise ratio and <10% CV in 384 well format.

TM3-Patched-Luc: TM3 cells transfected with Patched-Luc reporter. This cell line has ˜6-fold induction with relative high Luc signal and low % CV in 384 well format.

### Example 2

### Smoothened Interaction Assays

A Smo binding assay was developed using radio-labeled smoothened agonist for compound competition. An imaging-based or flow Cytometry-based system using Cy3-cyclopamine was developed for compound competition (Chen et al. (2002) Genes Dev 16: 2743). The assays were carried out for confirmed hits from reporter gene assays (RGAs) to identify compounds that target Smo directly.

Table 1, infra; lists the IC50 for displacement of a small molecule agonist of Smoothened determined in a filter binding format. Cyclopamine (Chen et al. (2002) Genes Dev 16: 2743), KAAD-cyclopamine (Chen et al. (2002) Genes Dev 16: 2743), SANT1 (Chen et al. (2002) PNAS 99: 14071) and Hh-Antag691 (Romer et al. (2004) Cancer Cell 6: 229) are reference compounds known to bind to Smoothened.

### Example 3

### Secondary Assays for Determining Cellular Function

The following secondary assays were applied to compound classes of interest:

“IC50 shift” assays in TMHh12 cells. IC50 for antagonism of gli-luciferase activity was tested in the presence of increasing concentrations of a small molecule agonist which binds to Smo with 1 nM affinity and activates the Hh pathway. Antagonist compounds from screening which show increased IC50s for gli-luc as the agonist dose is increased may be directly interacting with Smo (either through competition for the same binding site on Smo; or via competition between an active conformational state of Smo that is induced by agonist and an inactive state that is induced by the test antagonist). In validation experiments, a variety of small molecule antagonists of Smo demonstrate “IC50 shift” behavior.

Table 1 lists the IC50 of antagonists determined in the presence of different (1 nM and 25 nM) concentrations of a small agonist of Smoothened.

TABLE 1

| | Gli_luc (1 nM | Gli_luc (25 nM | Binding |
|---|---|---|---|
| | Smo agonist) | Smo agonist) | assay |
| Ex. | IC50 [nM] | IC50 [nM] | IC50 [nM] |
| 5 | <5 | 139 | 3 |
| 6 | 19 | 279 | 15 |
| 7 | <5 | 141 | 14 |
| 8 | <5 | 476 | |
| 9 | 39 | 876 | |
| 10 | 290 | 1340 | 137 |
| 11 | <5 | 371 | 14 |
| 12 | <5 | 141 | 4 |
| 13 | <5 | 136 | |
| 14 | <5 | 37 | 1 |
| 15 | <5 | 479 | 18 |
| 16 | 592 | 3702 | |
| 17 | 1370 | 15500 | |
| 18 | 39 | 876 | |
| 19 | | | |
| 20 | 94 | 964 | 151 |
| 21 | 254 | 646 | 1 |
| 22 | 456 | 3265 | 444 |
| 23 | 349 | 3040 | 93 |
| 24 | | | |
| 25 | 409 | 2496 | 90 |
| 26 | 35 | 458 | 8 |
| 27 | 12 | 605 | |
| 28 | 12 | 481 | 2 |
| 29 | 1669 | 4920 | 490 |
| 30 | 136 | 1440 | 39 |
| 31 | 281 | 3037 | 28 |
| 32 | 261 | 2861 | 130 |
| 33 | 4530 | 16416 | |
| 34 | | | |
| 35 | | | 208 |
| 36 | | | |
| 37 | 2981 | 10973 | |
| 38 | 1718 | 13257 | 6251 |
| 39 | 686 | 3721 | |
| 40 | 145 | 1913 | 445 |
| 41 | 82 | 616 | 3 |
| 42 | 2584 | | |
| 43 | | | |
| 44 | | | |
| 45 | 85 | 1143 | 141 |
| 46 | 831 | 3854 | |
| 47 | 1568 | 5963 | |
| 48 | | | |
| 49 | 226 | 2683 | 118 |
| 50 | 901 | 11874 | 621 |
| 51 | 7002 | 11223 | 13679 |
| 52 | 40 | 598 | |
| 53 | 261 | 1795 | 7907 |
| 54 | 8675 | 6803 | 16698 |
| 55 | 91 | 995 | |
| 56 | 165 | 4176 | 271 |
| 57 | 3063 | 17439 | 28 |
| 58 | 651 | 10092 | 4300 |
| 59 | 112 | 3493 | 21 |
| 60 | 13208 | 23621 | 3645 |
| 61 | 2097 | 11913 | 299 |
| 62 | 141 | 1628 | 103 |
| 63 | 686 | 13960 | 493 |
| 64 | 48 | 1309 | 135 |
| 65 | 85 | 4808 | 96 |
| 66 | 246 | 10000 | 593 |
| 67 | 142 | 4189 | 486 |
| 68 | 138 | 4859 | 367 |
| 69 | 2487 | 12609 | 619 |
| 70 | 50 | 504 | 24 |
| 71 | 5 | 235 | 11 |
| 72 | 38 | 667 | |
| 73 | 99 | 2241 | 936 |
| 74 | 19 | 557 | 47 |
| 75 | | | |
| 76 | 772 | 1250 | |
| 77 | 1412 | 12395 | |
| 78 | 1889 | | |
| 79 | 1327 | | |
| 80 | 3271 | | |
| 81 | 1332 | | |
| 82 | 1513 | 9028 | |
| 83 | 715 | 9734 | |
| 84 | 700 | 4483 | 881 |
| 85 | | | 12 |
| 86 | | | 265 |
| 87 | 2067 | 2580 | |
| 88 | 1393 | | |
| 89 | 202 | | |
| 90 | 5443 | | |
| 91 | 59 | 16308 | |
| 92 | 1014 | 1297 | 60 |
| 93 | 3391 | | |
| 94 | 6630 | | |
| 95 | 1418 | | |
| 96 | 1482 | | |
| 97 | 1364 | | |
| 98 | 324 | 24411 | |
| 99 | 1052 | | |
| 100 | 906 | | |
| 101 | 904 | 14110 | |
| 102 | 1899 | | 217 |
| 103 | 4775 | 10152 | 3201 |
| 104 | 3560 | | 3521 |
| 105 | 833 | 20418 | 338 |
| 106 | 5956 | | 330 |

### Example 4

### Compound Synthesis

The compounds of the invention can be prepared as described in PCT patent publications WO01/05767 and WO00/05201, and in Ksander, et al. (2001) Journal of Medicinal Chemistry, 44:4677, the contents of all of which are herein incorporated by reference. Enantiomerically pure 5-amino-2,3-dihydro-1H-inden-2-yl carbamic acid methyl ester intermediate needed for the preparation of compounds of Formula Id can be prepared as described in Prashad et al. (2001) Adv. Synth. Catal. 343, 461, the contents of which are herein incorporated by reference.

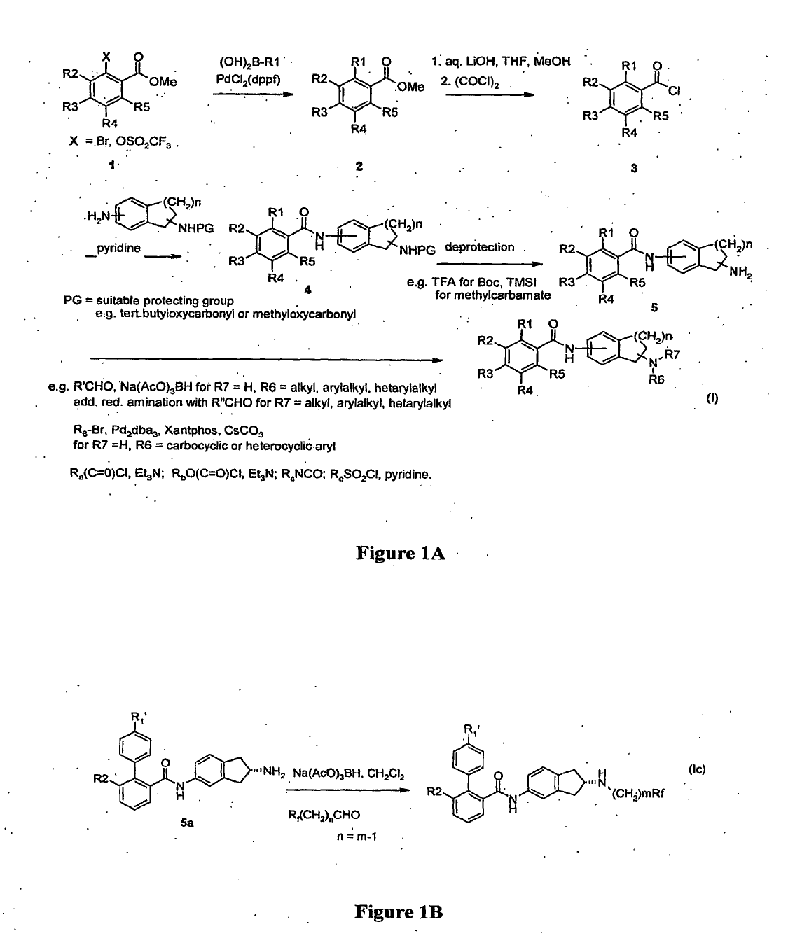

FIG. 1a shows a general synthetic scheme for the preparation of compounds of Formula I. Most preferred compounds of formula Ic can be prepared by reductive amination from intermediate 5a with aldehydes Rf(CH2)nCHO in the presence of a reducing agent such as sodium triacetoxy borohydride as shown in FIG. 1b.

### Examples 5-106

### GENERAL PROTOCOL FOR THE REDUCTIVE AMINATION OF 6-METHYL-4′-TRIFLUOROMETHYLBIPHENYL-2-CARBOXYLIC ACID ((S)-2-AMINO-INDAN-5-YL)AMIDE (EXAMPLES 7-34)

To 82 mg (0.2 mmol) of 6-methyl-4′-trifluoromethyl-biphenyl-2-carboxylic acid ((S)-2-amino-indan-5-yl)-amide and 67 mg (0.3 mmol, 1.5 eq.) sodium triacetoxy borohydride is added 2 ml of dichloromethane. Aldehyde (0.22 mmol, 1.1 eq.) is added and the mixture is stirred for 16 h at room temperature.

2 ml of sodium carbonate solution is added and the mixture is passed through a diatomeceous earth cartridge. After elution with additional dichloromethane, the organic phases are collected and evaporated. The residue is either dissolved in acetonitrile and methanol and purified by RP-HPLC or by flash chromatography.

### Example 5

### 6-Methyl-4′-trifluoromethyl-biphenyl-2-carboxylic acid {(S)-2-[(4-methyl-thiazol-2-ylmethyl)-amino]-indan-5-yl}-amide

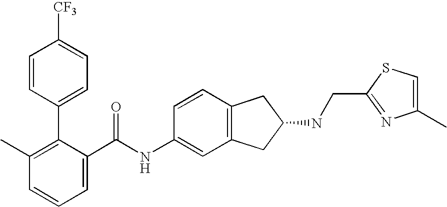

According to the general protocol, 37.7 mg of the above compound was isolated as a white powder.

¹H NMR (400 MHz, DMSO-D6) δ=2.54 (s, 3H) 2.76 (s, 3H) 3.03-3.1 (m, 2H) 3.27 (s, 1H) 3.43 (dd, J=15.41, 6.82 Hz, 2H) 3.95-4.03 (m, 1H) 4.41 (s, 2H) 7.44-7.49 (m, 1H) 7.52-7.56 (m, 2H) 7.70 (s, 1H) 7.84-7.94 (m, 5H) 8.18 (d, J=8.08 Hz, 2H) 10.43 (s, 1H).

HR-MS (m/z, MH+): meas. 522.1837 calc. 522.1827

### Example 6

### 6-Methyl-4′-trifluoromethyl-biphenyl-2-carboxylic acid {(S)-2-[(pyridin-2-ylmethyl)-amino]-indan-5-yl}-amide

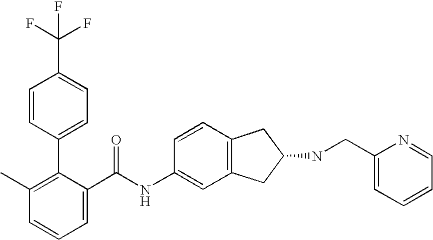

According to the general protocol 28 mg of the above compound were isolated as a white powder.

1H NMR (400 MHz, DMSO-D6) δ=2.55 (s, 3H) 3.08 (m, 2H) 3.42 (dd, J=15.41, 6.82 Hz, 2H) 3.91-4.00 (m, 1H) 4.28 (s, 2H) 7.44-7.49 (m, 1H) 7.51-7.57 (m, 1H) 7.65-7.74 (m, 2H) 7.85-7.96 (m, 6H) 8.18 (t, J=6.32 Hz, 3H) 8.94 (d, J=5.05 Hz, 1H).

HR-MS (m/z, MH+): meas. 502.2096 calc. 502.2106

### Examples 7-34

The following table (Table 2) lists examples of compounds prepared by reductive amination as described above:

TABLE 2

| Example | Structure | MS [m/z; M + 1] |
|---|---|---|
| 7 | 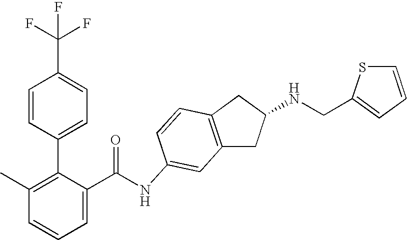 | 507 |
| 8 | 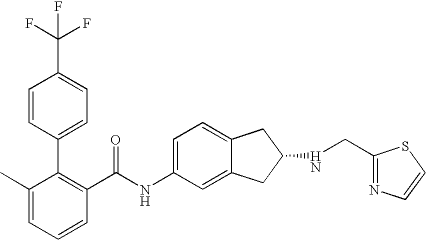 | 480 |
| 9 | 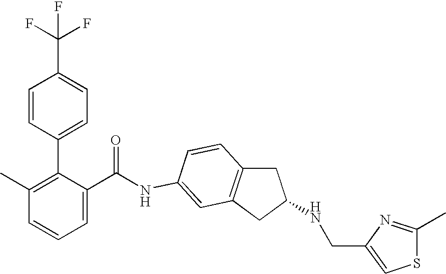 | 522 |
| 10 | 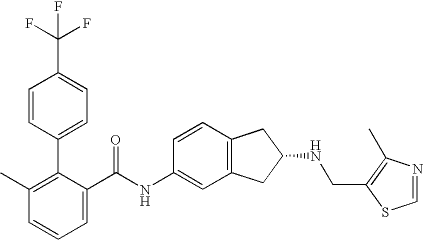 | 522 |
| 11 | 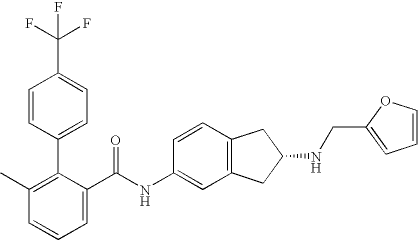 | 491 |
| 12 | 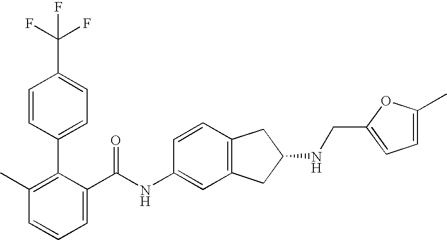 | 505 |
| 13 | 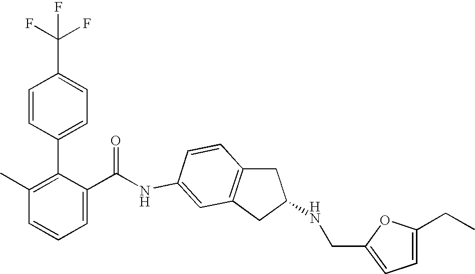 | 518 |
| 14 | 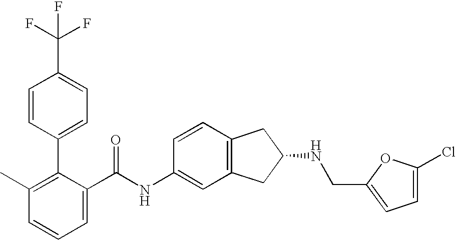 | 525 |
| 15 | 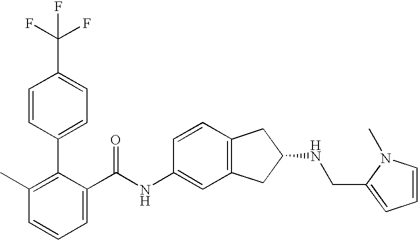 | 504 |
| 16 | 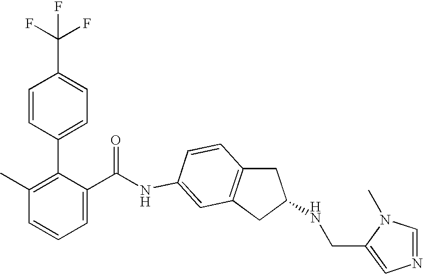 | 505 |
| 17 | 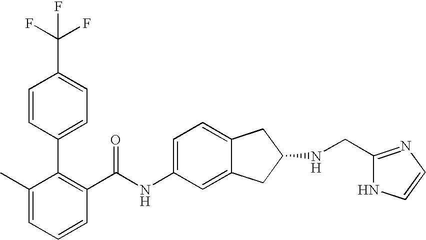 | 491 |
| 18 | 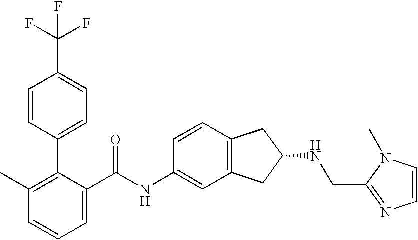 | 505 |
| 19 | 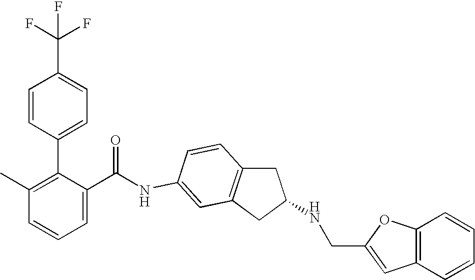 | 541 |
| 20 | 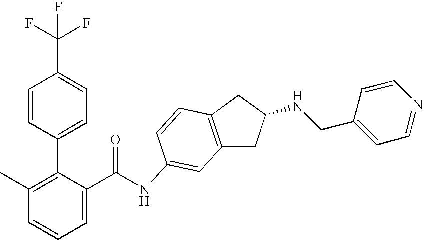 | 502 |
| 21 | 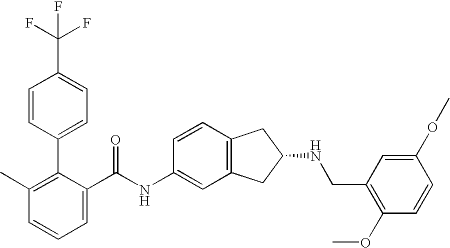 | 561 |
| 22 | 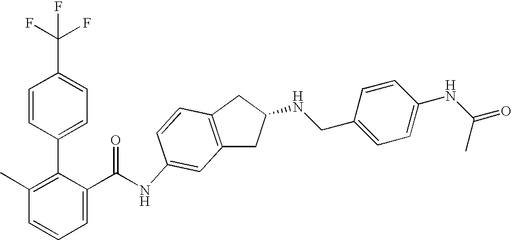 | 558 |
| 23 | 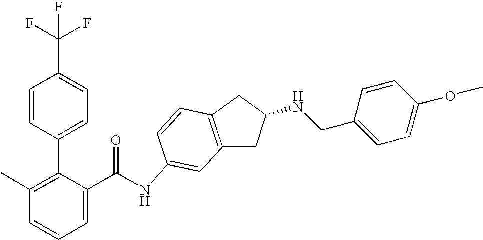 | 531 |
| 24 | 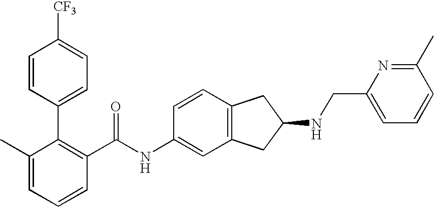 | 516 |
| 25 | 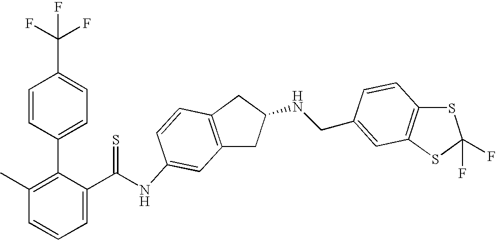 | 581 |
| 26 | 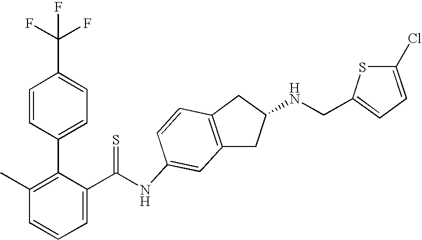 | 541 |
| 27 | 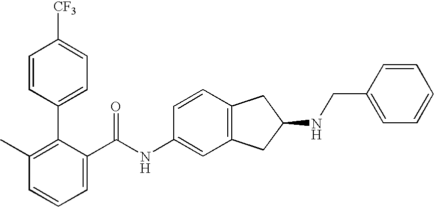 | 501 |
| 28 | 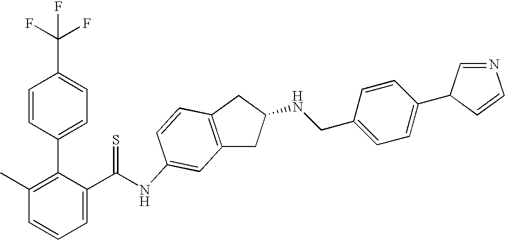 | 567 |
| 29 | 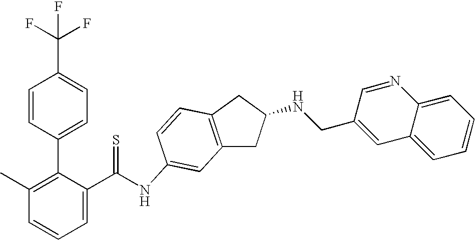 | 552 |
| 30 | 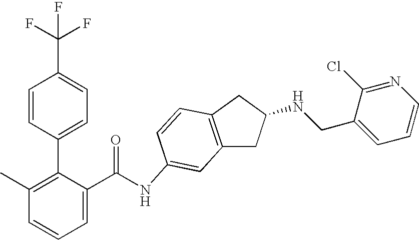 | 536 |
| 31 | 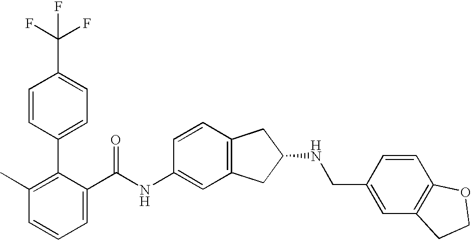 | 543 |
| 32 | 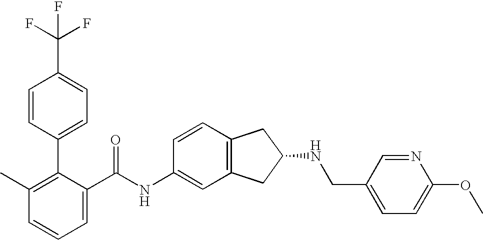 | 532 |
| 33 | 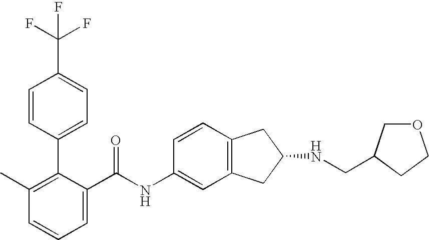 | 495 |
| 34 | 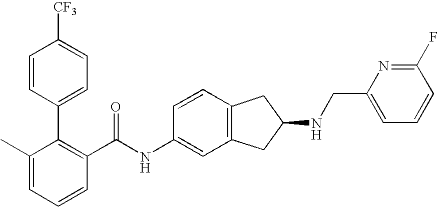 | 520 |

Further examples obtained from intermediate 5 by reductive amination (Table 3):

TABLE 3

| Ex. | Structure | MS (m/z, MH+) |
|---|---|---|
| 35 | 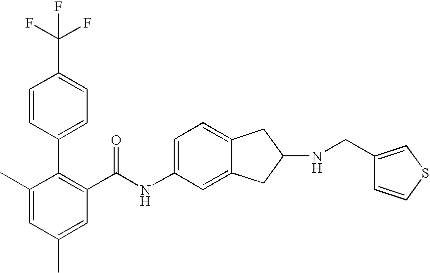 | 521 |
| 36 | 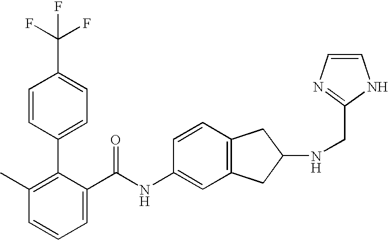 | 491 |
| 37 | 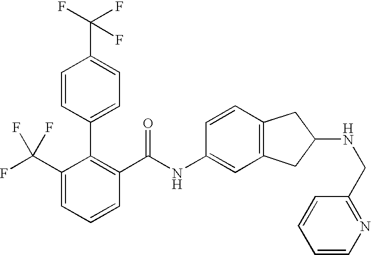 | 556 |
| 38 | 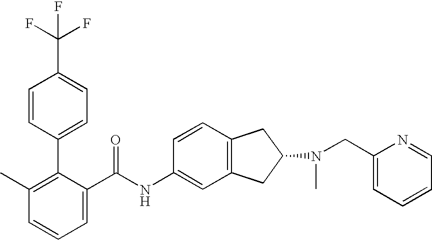 | 516 |
| 39 | 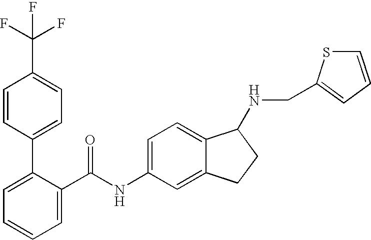 | 493 |
| 40 | 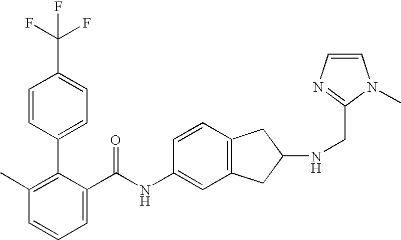 | 505 |
| 41 | 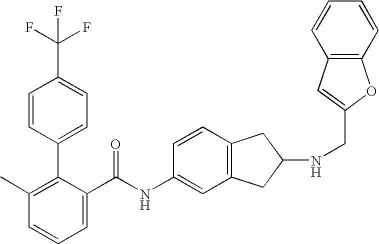 | 541 |
| 42 | 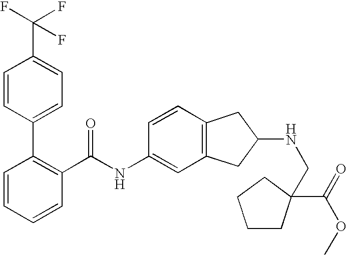 | 537 |
| 43 | 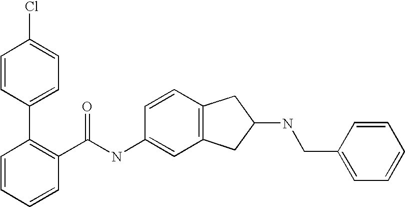 | 453 |
| 44 | 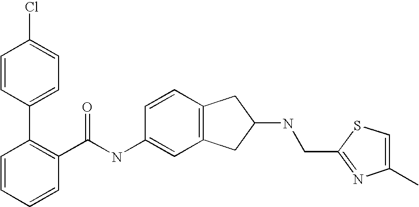 | 474 |
| 45 | 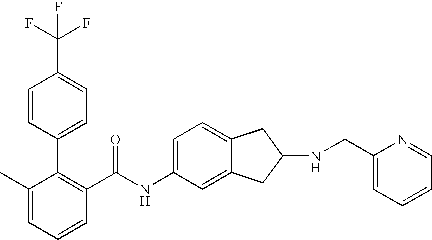 | 502 |
| 46 | 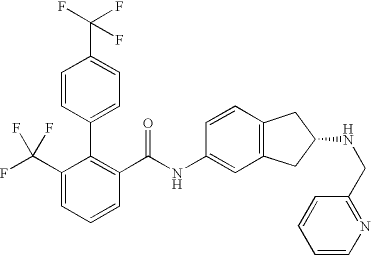 | 556 |
| 47 | 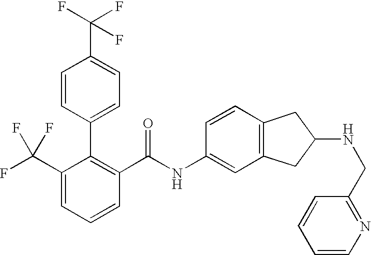 | 556 |
| 48 | 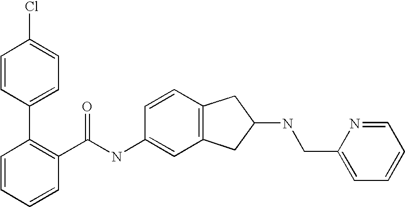 | 454 |
| 49 | 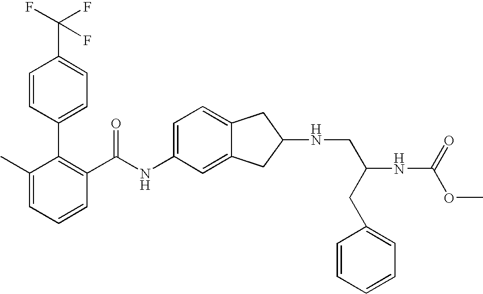 | 602 |
| 50 | 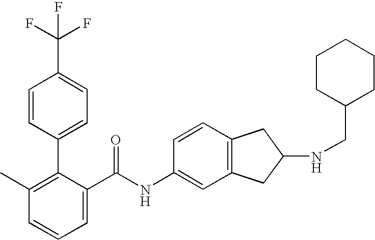 | 507 |
| 51 | 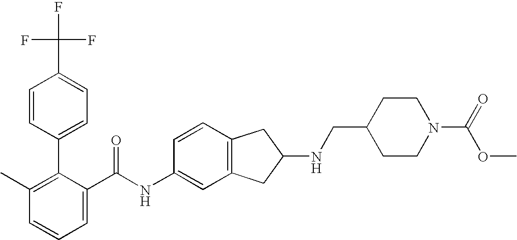 | 566 |
| 52 | 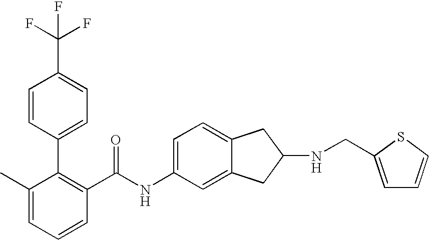 | 507 |
| 53 | 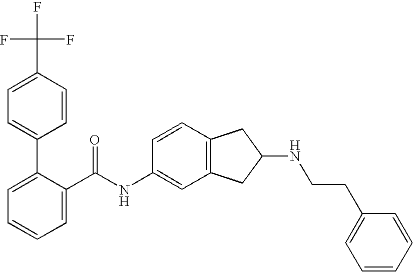 | 501 |
| 54 | 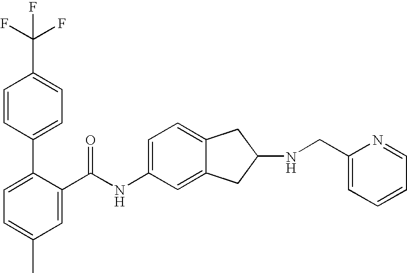 | 502 |
| 55 | 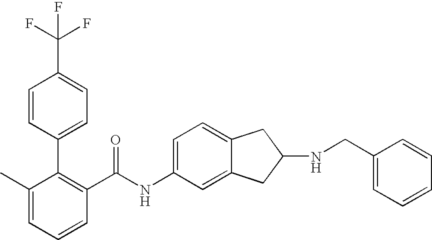 | 501 |
| 56 | 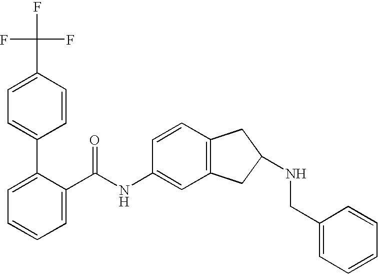 | 487 |
| 57 | 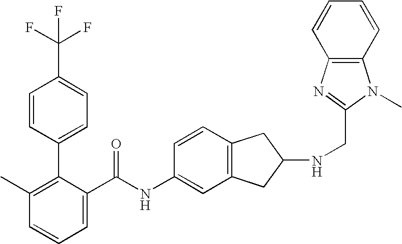 | 555 |
| 58 | 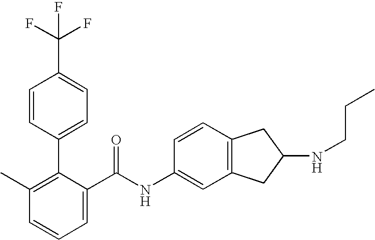 | 453 |
| 59 | 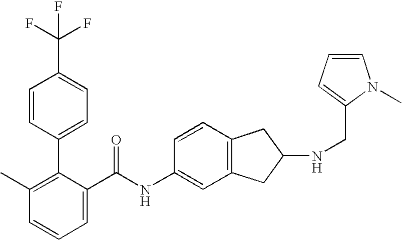 | 504 |
| 60 | 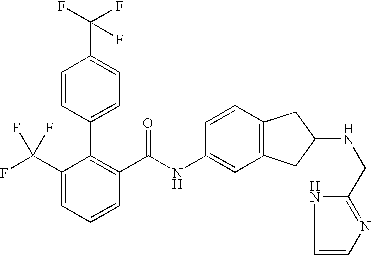 | 545 |
| 61 | 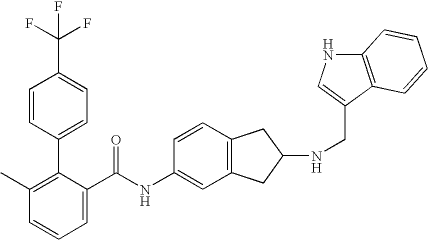 | 540 |
| 62 | 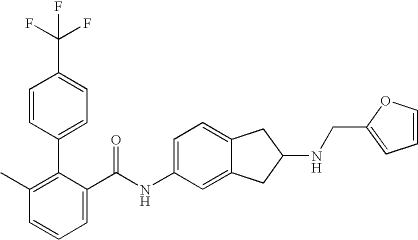 | 491 |
| 63 | 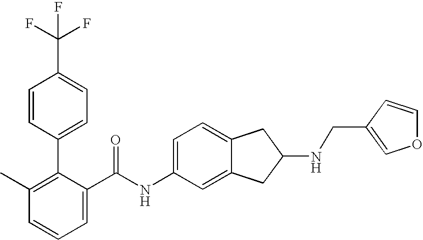 | 491 |
| 64 | 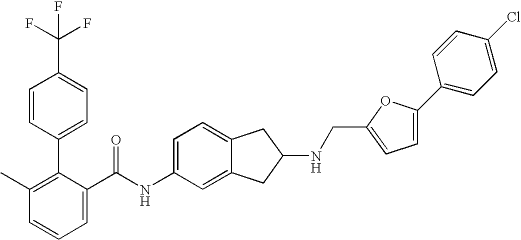 | 601 |
| 65 | 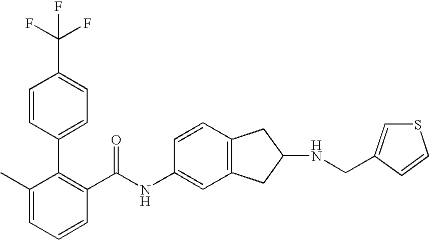 | 502 |
| 66 | 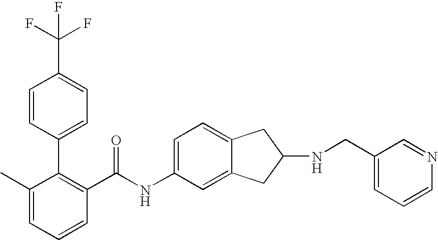 | 502 |
| 67 | 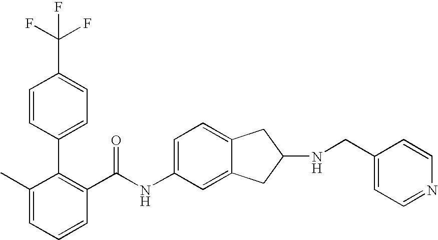 | 502 |
| 68 | 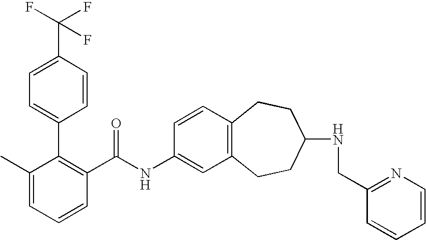 | 530 |
| 69 | 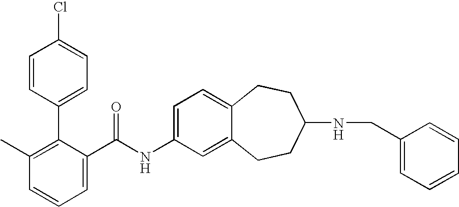 | 497 |
| 70 | 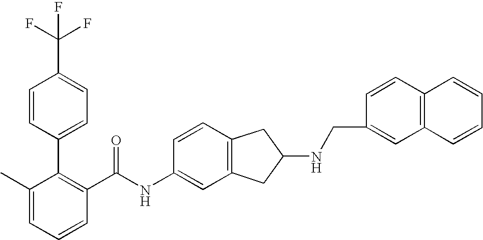 | 552 |
| 71 | 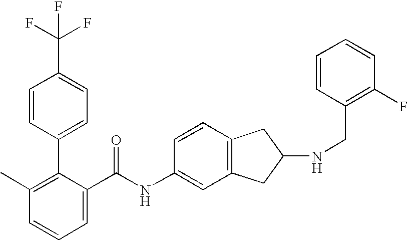 | 519 |
| 72 | 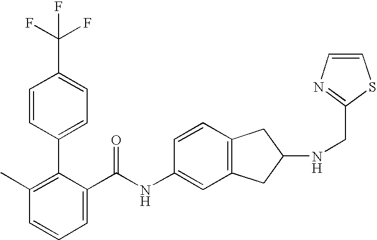 | 508 |
| 73 | 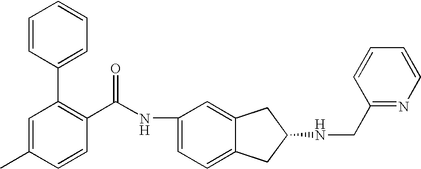 | 434 |

### Example 74

### 6-Methyl-4′-trifluoromethyl-biphenyl-2-carboxylic acid ((S)-2-phenylamino-indan-5-yl)-amide

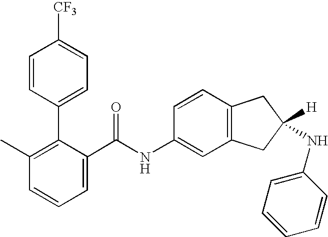

Bromobenzene (57 μL, 0.55 mmol, 1.5 eq.), 9.9-dimethyl-4,5-bis(diphenylphosphine)=Xanthphos (42 mg, 0.073 mmol, 0.2 eq.) and Cs2CO3 (167 mg, 0.51 mmol, 1.4 eq.) were mixed in a vial, followed by the addition of Pd2 dba3 (33.5 mg, 0.037 mmol, 0.1 eq.). The vial was purged with nitrogen and a solution of 6-methyl-4′-trifluoromethyl-biphenyl-2-carboxylic acid ((S)-2-amino-indan-5-yl)-amide (150 mg; 0.365 mmol, 1 eq.) in degassed toluene (1.8 ml) was added. The reaction mixture was heated in a microwave synthesizer for 1 h at 130° C. and additional 3 h at 140° C.

The mixture was filtered through Celite and concentrated. The residue was dissolved in methanol with some DMSO and purified by preparative HPLC to afford after removal of solvents 10 mg (6%) of the product.

1H NMR (400 MHz, DMSO-D6) δ=2.08 (S, 3H) 2.65-2.72 (m, 2H) 3.18 (ddd, J=115.79, 6.95, 3.03 Hz, 2H) 4.15 (d, J=6.57 Hz, 1H) 5.76 (d, J=6.57 Hz, 1H) 6.51 (t, J=7.07 Hz, 1H) 6.57 (d, J=7.58 Hz, 2H) 7.03-7.13 (m, 4H) 7.29 (s, 1H) 7.38-7.49 (m, 5H) 7.72 (d, J=8.08 Hz, 2H) 10.00 (s, 1H)

HR-MS (m/z, MH+) meas.: 487.2007 calc. 487.1997

### Example 75

### 6-Methyl-4′-trifluoromethyl-biphenyl-2-carboxylic acid [(S)-2-(pyridin-2-ylamino)-indan-5-yl]-amide

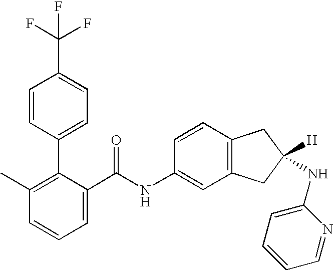

2-Bromopyridine (12 μL, 0.122 mmol, 1 eq.), rac-BINAP (3 mg), sodium tert-butanolate (23 mg, 0.244 mmol. 2 eq) were mixed in a vial, followed by the addition of Pd2dba3 (4.5 mg, 0.005 mmol, 0.04 eq.). The vial was purged with nitrogen and a solution of 6-methyl-4′-trifluoromethyl-biphenyl-2-carboxylic acid ((S)-2-amino-indan-5-yl)-amide (50 mg, 0.122 mmol, 1 eq.) in degassed toluene (1.1 ml) was added. The reaction mixture was heated at 60° C.

The crude reaction mixture was purified by preparative HPLC (10% to 100% acetonitrile in water with 3% isopropanol) to afford after removal of solvents 13 mg (22%) of the product.

MS: m/z 488 (MH+).

Table 4 contains examples obtained from intermediate 5 by acylation with acid chlorides:

TABLE 4

| Ex. | Structure | MS (m/z, MH+) |
|---|---|---|
| 76 | 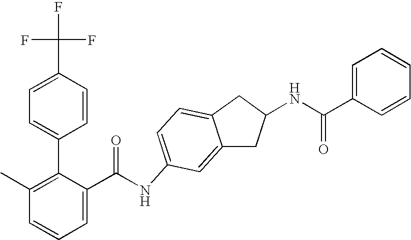 | 515 |
| 77 | 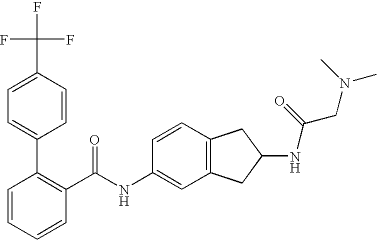 | 482 |
| 78 | 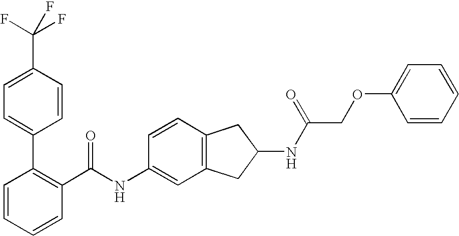 | 531 |
| 79 | 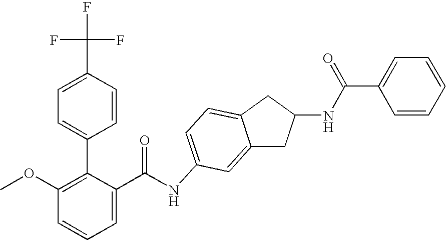 | 531 |
| 80 | 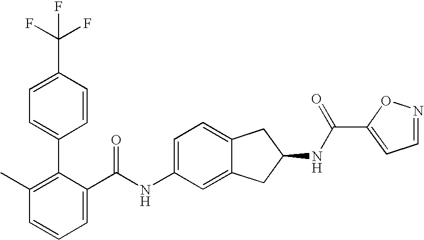 | 506 |

Table 5 contains examples obtained from intermediate 5 by acylation with chloro-formates:

TABLE 5

| Ex. | Structure | MS (m/z, MH+) |
|---|---|---|
| 81 | 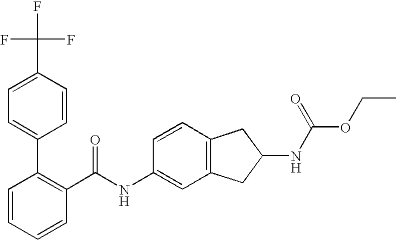 | 469 |
| 82 | 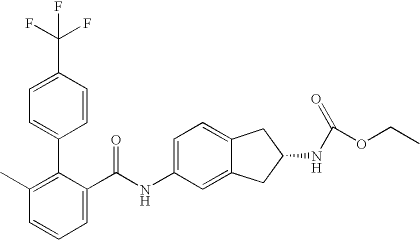 | 483 |
| 83 | 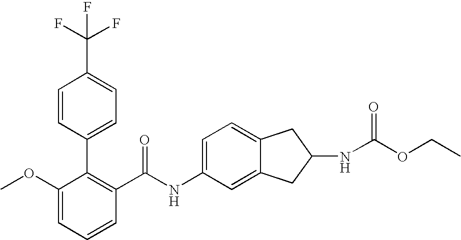 | 499 |
| 84 | 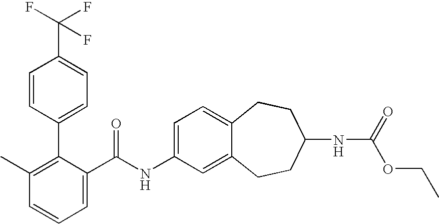 | 511 |

Table 6 contains examples obtained from intermediate 5 by sulfonylation with sulfonylchlorides:

TABLE 6

| Ex. | Structure | MS (m/z, MH+) |
|---|---|---|
| 85 | 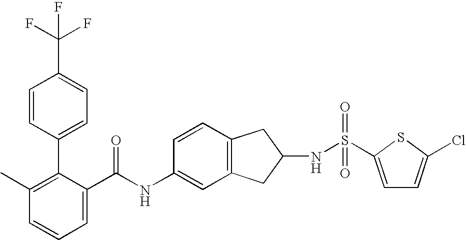 | 591 |
| 86 | 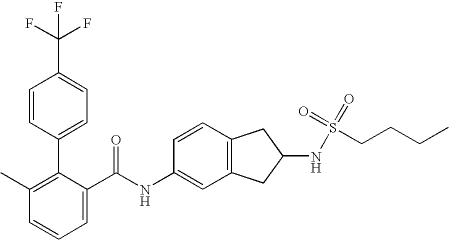 | 531 |
| 87 | 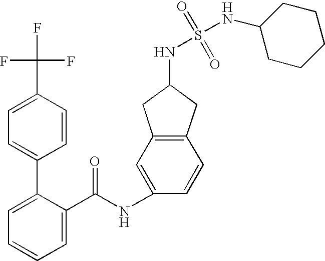 | 558 |
| 88 | 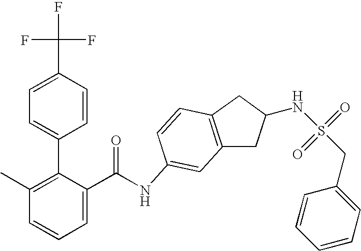 | 551 |
| 89 | 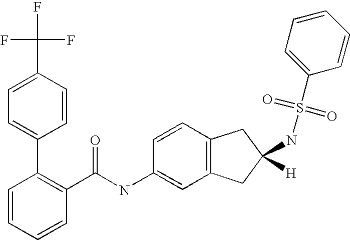 | 537 |
| 90 | 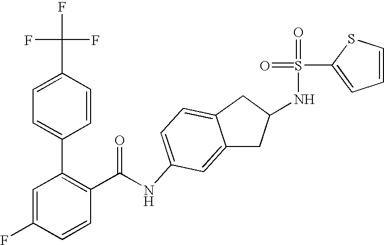 | 561 |
| 91 | 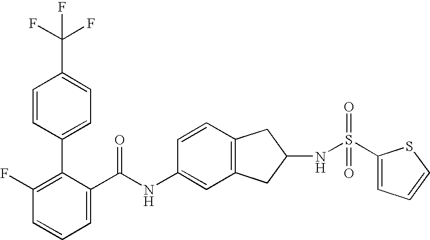 | 561 |
| 92 | 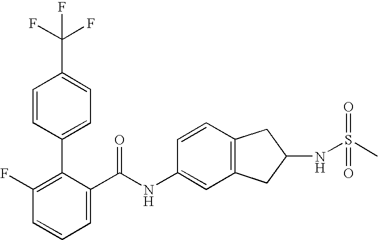 | 493 |
| 93 | 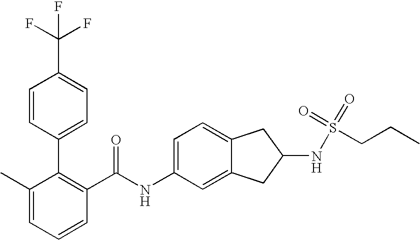 | 517 |
| 94 | 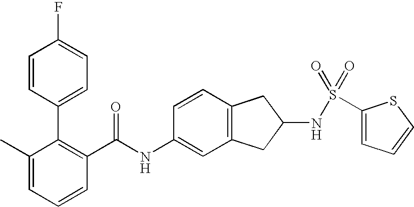 | 507 |
| 95 | 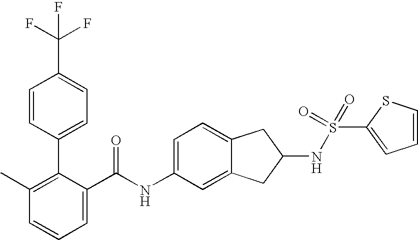 | 557 |
| 96 | 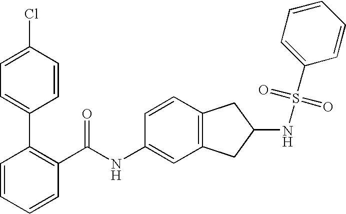 | 503 |
| 97 | 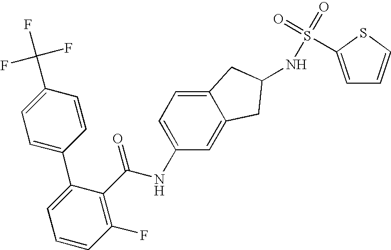 | 561 |
| 98 | 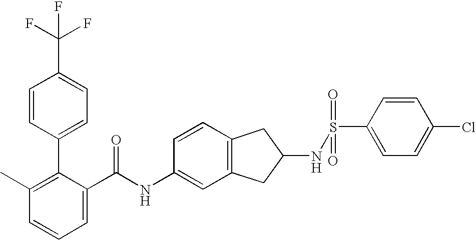 | 585 |
| 99 | 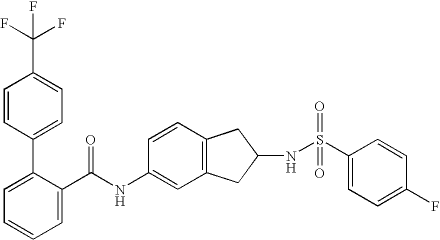 | 555 |
| 100 | 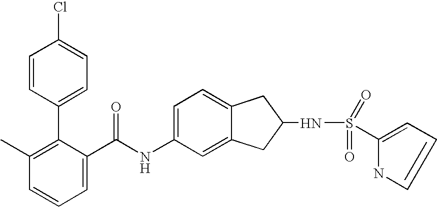 | 524 |
| 101 | 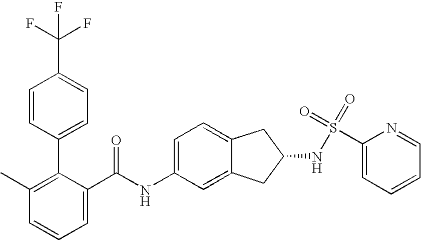 | 552 |
| 102 | 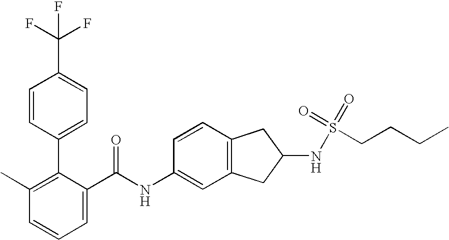 | 531 |
| 103 | 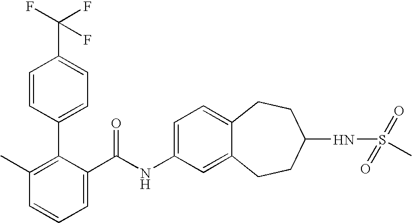 | 517 |
| 104 | 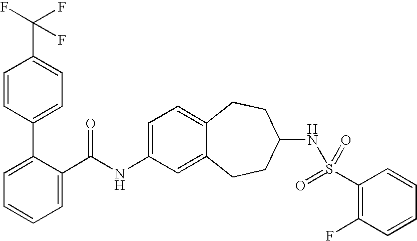 | 583 |
| 105 | 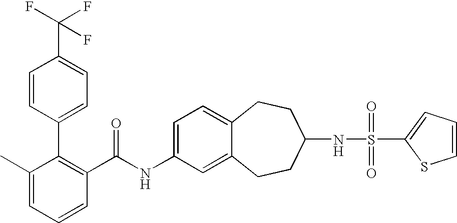 | 585 |
| 106 | 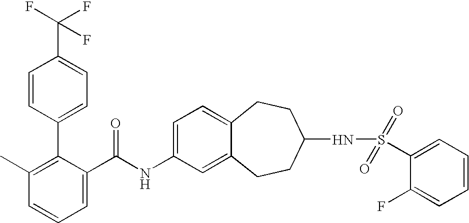 | 597 |

Compounds of the present invention are assayed to evaluate their capacity to inhibit the Hedgehog signaling pathway.

Gli-Luc Reporter Assay for Hh Pathway Inhibition

Gli-luciferase activity of TMHh12 cells was determined in the presence of Shh protein. Compounds of Formula I preferably have an EC₅₀ of less than 500 nM, more preferable less than 200 nM. The compound of example 6 has an EC₅₀ of 9.4 nM to block Shh-mediated pathway activation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method of treating a Hedgehog-related disorder comprising administering a compound of Formula (I) to a warm-blooded animal, especially a human, in need of such treatment: 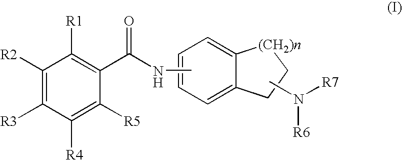
wherein:
R2-C, R3-C, R4-C or R5-C may be replaced by N
n is 1, 2 or 3
R1 is carbocyclic aryl or heteroaryl
R2, R3, R4 and R5 are independently hydrogen, lower alkyl, lower alkoxy, lower alkylthio, fluoro, chloro, bromo, amino, substituted amino, trifluoromethyl, acyloxy, alkylcarbonyl, trifluoromethoxy or cyano
R6 is hydrogen, optionally substituted alkyl, carbocyclic or heterocyclic aryl-lower alkyl
R7 is hydrogen, optionally substituted alkyl, carbocyclic aryl, heteroaryl, carbocyclic aryl-lower alkyl, heteroaryl-lower alkyl, or 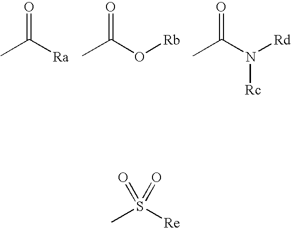
wherein
Ra is optionally substituted alkyl, cycloalkyl, aryl or heterocyclyl
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl, arylalkyl)
Re is optionally substituted alkyl, cycloalkyl, aryl or heterocyclyl, amino or substituted amino
and pharmaceutically acceptable salts thereof, and enantiomers thereof.

2. The method according to claim 1, further comprising administering a compound of Formula (Ia): 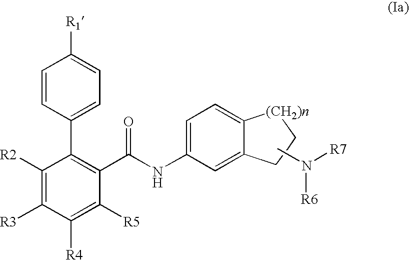
wherein R2-C, R3-C, R4-C or R5-C may be replaced by N; and
wherein
R1′ is hydrogen, fluoro, chloro, bromo, lower alkyl, cyano, methoxy, trifluoromethyl, trifluoromethoxy, dimethylamino
R2 to R7 have meaning as defined for Formula I,
and pharmaceutically acceptable salts thereof, and enantiomers thereof.

3. The method according to claim 1, further comprising administering compound of Formula (Ib): 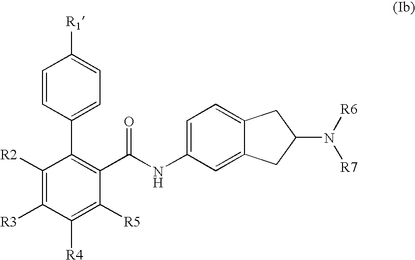
wherein:
R1′ is trifluoromethyl, chloro, fluoro
R2 and R3 are independently hydrogen, C1-C4 alkyl, C1-C4-alkoxy, trifluoromethyl, chloro or fluoro
R4 and R5 are hydrogen
R6 is hydrogen or C1-C3 alkyl
R7 is optionally substituted alkyl, carbocyclic aryl, heteroaryl, carbocyclic aryl-lower alkyl, heteroaryl-lower alkyl, or 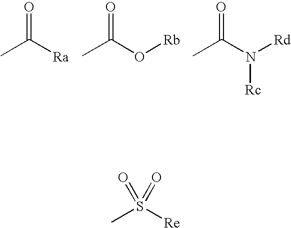
wherein
Ra is optionally substituted alkyl, cycloalkyl, aryl or heterocyclyl
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl, arylalkyl)
Re is optionally substituted alkyl, cycloalkyl, aryl or heterocyclyl, amino or substituted amino
and pharmaceutically acceptable salts thereof, and enantiomers thereof.

4. The method according to claim 1, further comprising administering compound of Formula (Ib)
wherein:
R1′ is trifluoromethyl, chloro, fluoro
R2 and R3 are independently hydrogen, C1-C4 alkyl, C1-C4-alkoxy, trifluoromethyl, chloro or fluoro
R4 and R5 are hydrogen
R6 is hydrogen
R7 is optionally substituted alkyl, carbocyclic aryl, heteroaryl, carbocyclic aryl-lower alkyl or, heteroaryl-lower alkyl
and pharmaceutically acceptable salts thereof, and enantiomers thereof.

5. The method according to claim 1, further comprising administering a compound of Formula (Ic): 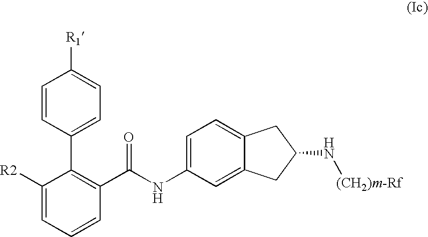
wherein:
R1′ is trifluoromethyl or chloro
R2 is hydrogen or methyl
m is 0 or 1;
Rf is carbocyclic or heterocyclic aryl
and pharmaceutically acceptable salts thereof.

6. The method according to claim 1, wherein the disease to be treated is a cancer.

7. The method according to claim 1, wherein the disease to be treated is benign prostate hyperplasia, psoriasis, wet macular degeneration, or osteoporosis.

8. A method of inhibiting Smo-dependent pathway activation comprising administering a compound of Formula (I) to a warm-blooded animal, especially a human: 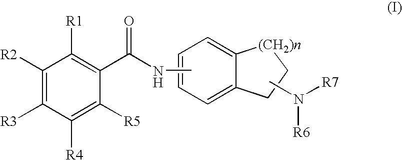
wherein:
R2-C, R3-C, R4-C or R5-C may be replaced by N
n is 1, 2 or 3
R1 is carbocyclic aryl or heteroaryl
R2, R3, R4 and R5 are independently hydrogen, lower alkyl, lower alkoxy, lower alkylthio, fluoro, chloro, bromo, amino, substituted amino, trifluoromethyl, acyloxy, alkylcarbonyl, trifluoromethoxy or cyano
R6 is hydrogen, optionally substituted alkyl, carbocyclic or heterocyclic aryl-lower alkyl
R7 is hydrogen, optionally substituted alkyl, carbocyclic aryl, heteroaryl, carbocyclic aryl-lower alkyl, heteroaryl-lower alkyl, or 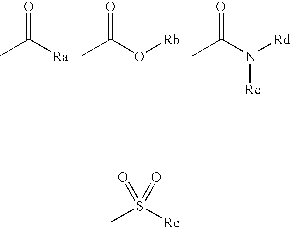
wherein
Ra is optionally substituted alkyl, cycloalkyl, aryl or heterocyclyl
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl, arylalkyl);
Re is optionally substituted alkyl, cycloalkyl, aryl or heterocyclyl, amino or substituted amino
and pharmaceutically acceptable salts thereof, and enantiomers thereof.

9. The method according to claim 8, further comprising administering a compound of Formula (Ia): 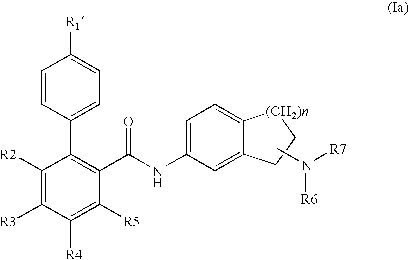
wherein R2-C, R3-C, R4-C or R5-C may be replaced by N; and
wherein
R1′ is hydrogen, fluoro, chloro, bromo, lower alkyl, cyano, methoxy, trifluoromethyl, trifluoromethoxy, dimethylamino
R2 to R7 have meaning as defined for Formula I,
and pharmaceutically acceptable salts thereof, and enantiomers thereof.

10. The method according to claim 8, further comprising administering compound of Formula (Ib): 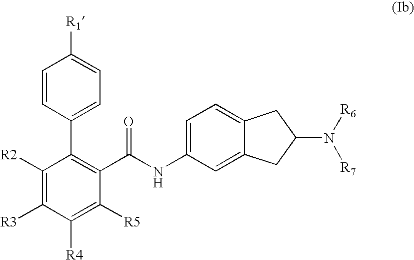
wherein:
R1′ is trifluoromethyl, chloro, fluoro
R2 and R3 are independently hydrogen, C1-C4 alkyl, C1-C4-alkoxy, trifluoromethyl, chloro or fluoro
R4 and R5 are hydrogen
R6 is hydrogen or C1-C3 alkyl;
R7 is optionally substituted alkyl, carbocyclic aryl, heteroaryl, carbocyclic aryl-lower alkyl, heteroaryl-lower alkyl, or 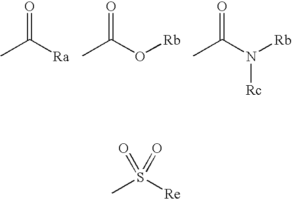
wherein
Ra is optionally substituted alkyl, cycloalkyl, aryl or heterocyclyl
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl; arylalkyl)
Re is optionally substituted alkyl, cycloalkyl, aryl or heterocyclyl, amino or substituted amino
and pharmaceutically acceptable salts thereof, and enantiomers thereof.

11. The method according to claim 8, further comprising administering compound of Formula (Ib)
wherein:
R1′ is trifluoromethyl, chloro, fluoro
R2 and R3 are independently hydrogen, C1-C4 alkyl, C1-C4-alkoxy, trifluoromethyl, chloro or fluoro
R4 and R5 are hydrogen
R6 is hydrogen
R7 is optionally substituted alkyl, carbocyclic aryl, heteroaryl, carbocyclic aryl-lower alkyl, heteroaryl-lower alkyl
and pharmaceutically acceptable salts thereof, and enantiomers thereof.

12. The method according to claim 8, further comprising administering a compound of Formula (Ic): 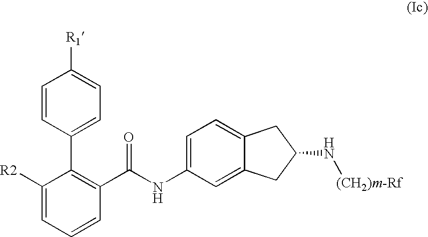
wherein:
R1′ is trifluoromethyl or chloro
R2 is hydrogen or methyl
m is 0 or 1
Rf is carbocyclic or heterocyclic aryl
and pharmaceutically acceptable salts thereof.

13. The method of claim 8, wherein the warm-blooded animal has cancer.

14. The method of claim 8, wherein the warm-blooded animal suffers from benign prostate hyperplasia, psoriasis, wet macular degeneration, or osteoporosis.

15. A method of regulating cellular proliferation or differentiation comprising administering a compound of Formula (I) to a warm-blooded animal, especially a human: 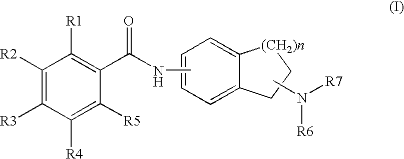
wherein:
R2-C, R3-C, R4-C or R5-C may be replaced by N
n is 1, 2 or 3
R1 is carbocyclic aryl or heteroaryl
R2, R3, R4 and R5 are independently hydrogen, lower alkyl, lower alkoxy, lower alkylthio, fluoro, chloro, bromo, amino, substituted amino, trifluoromethyl, acyloxy, alkylcarbonyl, trifluoromethoxy or cyano
R6 is hydrogen, optionally substituted alkyl, carbocyclic or heterocyclic aryl-lower alkyl
R7 is hydrogen, optionally substituted alkyl, carbocyclic aryl, heteroaryl, carbocyclic aryl-lower alkyl, heteroaryl-lower alkyl, or 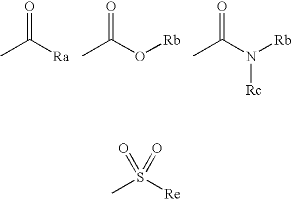
wherein
Ra is optionally substituted alkyl, cycloalkyl, aryl or heterocyclyl;
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl, arylalkyl)
Re is optionally substituted alkyl, cycloalkyl, aryl or heterocyclyl, amino or substituted amino
and pharmaceutically acceptable salts thereof, and enantiomers thereof.

16. The method according to claim 15, further comprising administering a compound of Formula (Ia): 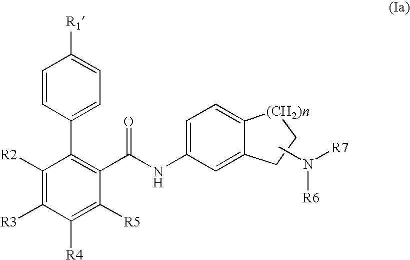
wherein R2-C, R3-C, R4-C or R5-C may be replaced by N; and
wherein
R1′ is hydrogen, fluoro, chloro, bromo, lower alkyl, cyano, methoxy, trifluoromethyl, trifluoromethoxy, dimethylamino
R2 to R7 have meaning as defined for Formula I,
and pharmaceutically acceptable salts thereof, and enantiomers thereof.

17. The method according to claim 15, further comprising administering compound of Formula (Ib): 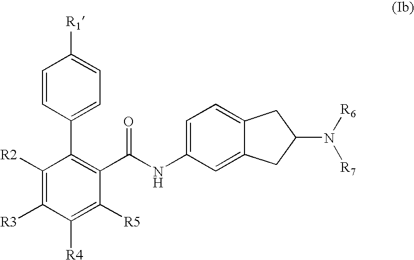
wherein:
R1′ is trifluoromethyl, chloro, fluoro;
R2 and R3 are independently hydrogen, C1-C4 alkyl, C1-C4-alkoxy, trifluoromethyl, chloro or fluoro
R4 and R5 are hydrogen
R6 is hydrogen or C1-C3 alkyl
R7 is optionally substituted alkyl, carbocyclic aryl, heteroaryl, carbocyclic aryl-lower alkyl, heteroaryl-lower alkyl, or 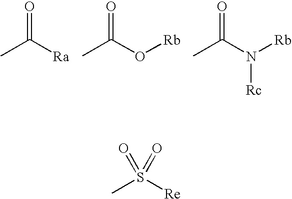
wherein
Ra is optionally substituted alkyl, cycloalkyl, aryl or heterocyclyl
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl, arylalkyl)
Re is optionally substituted alkyl, cycloalkyl, aryl or heterocyclyl, amino or substituted amino
and pharmaceutically acceptable salts thereof, and enantiomers thereof.

18. The method according to claim 15, further comprising administering compound of Formula (Ib)
wherein:
R1′ is trifluoromethyl, chloro, fluoro
R2 and R3 are independently hydrogen, C1-C4 alkyl, C1-C4-alkoxy, trifluoromethyl, chloro or fluoro
R4 and R5 are hydrogen
R6 is hydrogen
R7 is optionally substituted alkyl, carbocyclic aryl, heteroaryl, carbocyclic aryl-lower alkyl or, heteroaryl-lower alkyl
and pharmaceutically acceptable salts thereof, and enantiomers thereof.

19. The method according to claim 15, further comprising administering a compound of Formula (Ic): 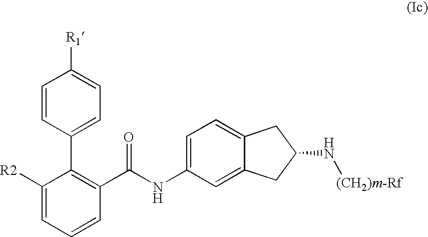
wherein:
R1′ is trifluoromethyl or chloro
R2 is hydrogen or methyl
m is 0 or 1
Rf is carbocyclic or heterocyclic aryl
and pharmaceutically acceptable salts thereof.

20. The method of claim 15, wherein the warm-blooded animal has cancer.

21. The method of claim 15, wherein the warm-blooded animal suffers from benign prostate hyperplasia, psoriasis, wet macular degeneration, or osteoporosis.

22. A method of treating a Hedgehog-related disorder comprising administering a pharmaceutical composition comprising a compound of Formula (I) to a warm-blooded animal, especially a human, in need of such treatment: 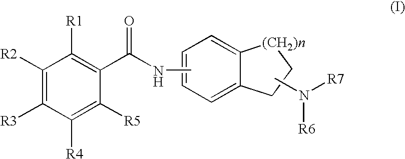
wherein:
R2-C, R3-C, R4-C or R5-C may be replaced by N
n is 1, 2 or 3
R1 is carbocyclic aryl or heteroaryl
R2, R3, R4 and R5 are independently hydrogen, lower alkyl, lower alkoxy, lower alkylthio, fluoro, chloro, bromo, amino; substituted amino, trifluoromethyl, acyloxy, alkylcarbonyl, trifluoromethoxy or cyano
R6 is hydrogen, optionally substituted alkyl, carbocyclic or heterocyclic aryl-lower alkyl;
R7 is hydrogen, optionally substituted alkyl, carbocyclic aryl, heteroaryl, carbocyclic aryl-lower alkyl, heteroaryl-lower alkyl, or 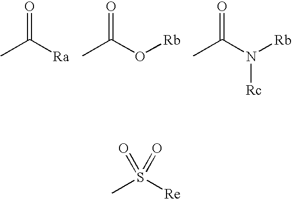
wherein
Ra is optionally substituted alkyl, cycloalkyl, aryl or heterocyclyl
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl, arylalkyl)
Re is optionally substituted alkyl, cycloalkyl, aryl or heterocyclyl, amino or substituted amino
and pharmaceutically acceptable salts thereof, and enantiomers thereof.

23. The method according to claim 22, further comprising administering a compound of Formula (Ia): 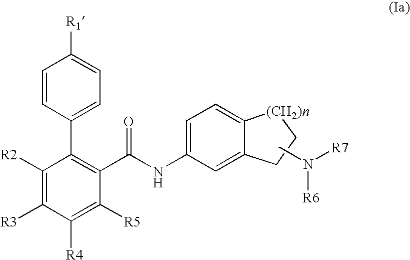
wherein R2-C, R3-C, R4-C or R5-C may be replaced by N; and
wherein
R1′ is hydrogen, fluoro, chloro, bromo, lower alkyl, cyano, methoxy, trifluoromethyl, trifluoromethoxy, dimethylamino
R2 to R7 have meaning as defined for Formula I,
and pharmaceutically acceptable salts thereof, and enantiomers thereof.

24. The method according to claim 22, further comprising administering compound of Formula (Ib): 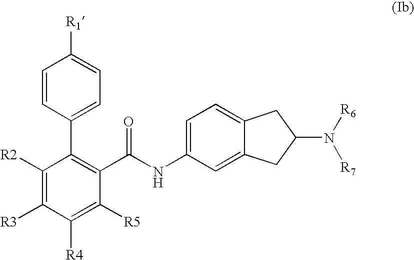
wherein:
R1′ is trifluoromethyl, chloro, fluoro
R2 and R3 are independently hydrogen, C1-C4 alkyl, C1-C4-alkoxy, trifluoromethyl, chloro or fluoro
R4 and R5 are hydrogen
R6 is hydrogen or C1-C3 alkyl
R7 is optionally substituted alkyl, carbocyclic aryl, heteroaryl, carbocyclic aryl-lower alkyl, heteroaryl-lower alkyl, or 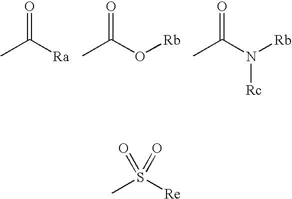
wherein,
Ra is optionally substituted alkyl, cycloalkyl, aryl or heterocyclyl
Rb is optionally substituted alkyl, cycloalkyl, aryl or heterocyclyl
Rc and Rd are independently hydrogen, substituted alkyl, cycloalkyl, aryl; or heterocyclyl, or Rc and Rd together represent lower alkylene or lower alkylene interrupted by O, S, N—(H, alkyl, arylalkyl)
Re is optionally substituted alkyl, cycloalkyl, aryl or heterocyclyl, amino or substituted amino
and pharmaceutically acceptable salts thereof, and enantiomers thereof.

25. The method according to claim 22, further comprising administering compound of Formula (Ib)
wherein:
R1′ is trifluoromethyl, chloro, fluoro
R2 and R3 are independently hydrogen, C1-C4 alkyl, C1-C4-alkoxy, trifluoromethyl, chloro or fluoro
R4 and R5 are hydrogen;
R6 is hydrogen
R7 is optionally substituted alkyl, carbocyclic aryl, heteroaryl, carbocyclic aryl-lower alkylheteroaryl-lower alkyl
and pharmaceutically acceptable salts thereof, and enantiomers thereof.

26. The method according to claim 22, further comprising administering a compound of Formula (Ic): 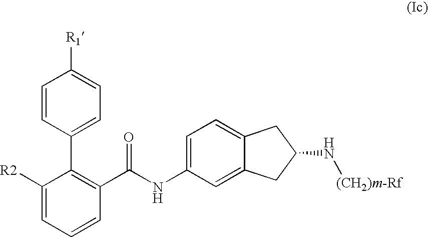
wherein:
R1′ is trifluoromethyl or chloro
R2 is hydrogen or methyl
m is 0 or 1
Rf is carbocyclic or heterocyclic aryl
and pharmaceutically acceptable salts thereof.

27. The method of claim 22, wherein the warm-blooded animal has cancer.

28. The method of claim 22, wherein the warm-blooded animal suffers from benign prostate hyperplasia, psoriasis, wet macular degeneration, or osteoporosis.
